(19) United States
(12) Patent Application Publication
(10) Pub. No.: US 2022/0354880 A1
(43) Pub. Date: Nov. 10, 2022

(54) COMBINATION TREATMENT FOR CANCER

(71) Applicant: AstraZeneca AB, Södertälje (SE)

(72) Inventor: Theresa PROIA, Waltham (UA)

(21) Appl. No.: 17/754,258

(22) PCT Filed: Sep. 29, 2020
(86) PCT No.: PCT/IB2020/059097

§ 371 Date: Mar. 29, 2022

Related U.S. Application Data
(60) Provisional application No. 62/907,773, filed on Sep. 30, 2019.

(51) Int. Cl. A61K 31/7088; A61K 39/395; A61K 33/243; A61P 35/00

(52) U.S. Cl. CPC A61K 31/7088; A61K 39/3955; A61K 33/243; A61P 35/00

(57) ABSTRACT

The present disclosure provides methods of treating cancer in a patient, comprising administering to the patient a chemotherapeutic agent, an immunomodulatory agent, and an antisense compound targeted to STAT3. Also provided herein are compositions and kits for performing the methods provided herein. In preferred embodiments, the chemotherapeutic agent is cisplatin, the antisense compound targeted to STAT3 is AZD9150, and the immunomodulatory agent is MEDI4736.

### FIELD OF THE DISCLOSURE

The present disclosure provides methods of treating cancer in a patient, comprising administering to the patient a chemotherapeutic agent, an immunomodulatory agent, and an antisense compound targeted to STAT3. Also provided herein are compositions and kits for performing the methods provided herein.

### BACKGROUND

Chemotherapy-immunotherapy (“chemo-IO”) combinations are being explored as a potentially powerful cancer treatment tool. The combination of antisense compounds, e.g., targeted to the master immune regulator STAT3, and immunomodulatory agents, e.g., immune checkpoint inhibitors, for immunotherapy is described in WO 2016/062722. Immune responses mediated by immune checkpoint inhibition may be enhanced by the immunogenic effects of cytotoxic agents, which as a result of direct tumor cell killing, can increase tumor antigens. Chemo-IO combination strategies can minimize direct T cell killing with chemotherapy, enhance antigen presentation, and promote T cell activation. Challenges associated with development of a chemo-IO combination treatment can include finding an effective combination of agents and determining effective dosing amounts and schedules, since a drug combination will likely affect a patient differently than merely providing an additive effect of each drug alone. Furthermore, many current chemotherapy agents have many adverse side effects, and a further challenge in development is therefore to decrease the side effects of a combination therapy, while increasing effectiveness, compared with each drug alone.

### SUMMARY OF THE DISCLOSURE

In some embodiments, the present disclosure provides a method of treating cancer in a patient comprising administering to the patient: (a) about 50 mg/m² to about 70 mg/m² chemotherapeutic agent; (b) an immunomodulatory agent; and (c) an antisense compound targeted to STAT3.

In some embodiments, the immunomodulatory agent is an immune checkpoint inhibitor. In some embodiments, the immunomodulatory agent is selected from an anti-PD-L1 antibody or antigen-binding fragment thereof; an anti-PD1 antibody or antigen-binding fragment thereof; an anti-CTLA-4 antibody or antigen-binding fragment thereof; and an OX-40 agonist. In some embodiments, the immunomodulatory agent is selected from MEDI4736, MPDL3280A, 2.7A4, AMP-714, MDX-1105, nivolumab, pembrolizumab, pidilizumab, BMS936559, MPDL3280A, tremelimumab, ipilimumab and OX40L FP. In some embodiments, the immunomodulatory agent is an anti-PD-L1 antibody. In some embodiments, the anti-PD-L1 antibody is MEDI4736.

In some embodiments, the antisense compound targeted to STAT3 does not inhibit STAT1, STAT4, or STAT6. In some embodiments, the antisense compound targeted to STAT3 is an antisense oligonucleotide. In some embodiments, the antisense compound targeted to STAT3 is AZD9150.

In some embodiments, the immunomodulatory agent is MEDI4736 or an antigen-binding fragment thereof, and the antisense compound targeted to STAT3 is AZD9150.

In some embodiments, the method comprises administering about 1 mg/kg to about 20 mg/kg MEDI4736 or an antigen-binding fragment thereof. In some embodiments, the method comprises administering about 200 mg to about 400 mg AZD9150.

In some embodiments, the chemotherapeutic agent administered to the patient is cisplatin. In some embodiments, the method comprises administering about 55 mg/m² to about 65 mg/m² cisplatin. In some embodiments, the method comprises administering 60 mg/m² cisplatin.

In some embodiments, the cancer is selected from breast cancer, renal carcinoma, lung cancer, pancreatic cancer, colorectal cancer, hepatocellular carcinoma (HCC), head and neck cancer, and lymphoma. In some embodiments, the lung cancer is non-small cell lung cancer (NSCLC). In some embodiments, the head and neck cancer is head and neck squamous cell carcinoma (HNSCC). In some embodiments, the lymphoma is diffuse large B-cell carcinoma (DLBCL).

In some embodiments, the patient has a PD-L1 positive cancer. In some embodiments, the patient comprises cancer cells expressing PD-L1.

In some embodiments, in a treatment cycle, the chemotherapeutic agent, the immunomodulatory agent, and the antisense compound targeted to STAT3 are administered to the patient concurrently. In some embodiments, in a treatment cycle, the chemotherapeutic agent is administered to the patient before the immunomodulatory agent and the antisense compound targeted to STAT3. In some embodiments, in a treatment cycle, the chemotherapeutic agent and the immunomodulatory agent are administered to the patient before the antisense compound targeted to STAT3.

In some embodiments, in a treatment cycle, fewer doses of the chemotherapeutic agent are administered to the patient than the immunomodulatory agent and the antisense compound targeted to STAT3. In some embodiments, in a treatment cycle, about 1 dose of the chemotherapeutic agent, about 2 to about 5 doses of the immunomodulatory agent, and about 5 to about 20 doses of the antisense compound targeted to STAT3 are administered to the patient.

In some embodiments, the treatment cycle is one week, two weeks, three weeks, or four weeks. In some embodiments, the method comprises two to eight treatment cycles.

In some embodiments, the method results in an increase in CD11b+/Ly6C+ dendritic cells compared to administration of the immunomodulatory agent alone, administration of the antisense compound targeted to STAT3 alone, or administration of the chemotherapeutic agent alone.

In some embodiments, the method results in an increase in progression-free survival and/or overall survival as compared to administration of the immunomodulatory agent alone, administration of the antisense compound targeted to STAT3 alone, or administration of the chemotherapeutic agent alone.

In some embodiments, the present disclosure provides a method of treating cancer in a patient comprising administering to the patient: (a) about 50 mg/m2 to about 60 mg/m² cisplatin; (b) about 1 mg/kg to about 20 mg/kg MEDI4736; and (c) about 200 mg to about 400 mg AZD9150.

In some embodiments, the method comprises administering about 60 mg/m² cisplatin, about 10 mg/kg MEDI4736, and about 300 mg AZD9150.

In some embodiments, the present disclosure provides a pharmaceutical composition comprising: (a) a chemotherapeutic agent; and (b) an immunomodulatory agent, wherein the chemotherapeutic agent and the immunomodulatory agent are in the pharmaceutical composition at a weight ratio of about 1:1 to about 1:4.

In some embodiments, the chemotherapeutic agent is cisplatin. In some embodiments, the immunomodulatory agent is an immune checkpoint inhibitor.

In some embodiments, the immunomodulatory agent is selected from an anti-PD-L1 antibody or antigen-binding fragment thereof; an anti-PD1 antibody or antigen-binding fragment thereof; an anti-CTLA-4 antibody or antigen-binding fragment thereof; and an OX-40 agonist.

In some embodiments, the immunomodulatory agent is selected from MEDI4736, MPDL3280A, 2.7A4, AMP-714, MDX-1105, nivolumab, pembrolizumab, pidilizumab, BMS936559, MPDL3280A, tremelimumab, ipilimumab, and OX40L FP.

In some embodiments, the immunomodulatory agent is an anti-PD-L1 antibody. In some embodiments, the anti-PD-L1 antibody is MEDI4736.

In some embodiments, the chemotherapeutic agent and the immunomodulatory agent are in the pharmaceutical composition at a weight ratio of about 1:2.

In some embodiments, the present disclosure provides a kit for treating cancer, comprising: (a) an chemotherapeutic agent; (b) an immunomodulatory agent; and (c) an antisense compound targeted to STAT3.

In some embodiments, the chemotherapeutic agent is cisplatin. In some embodiments, the immunomodulatory agent is an immune checkpoint inhibitor. In some embodiments, the immunomodulatory agent is selected from an anti-PD-L1 antibody or antigen-binding fragment thereof; an anti-PD1 antibody or antigen-binding fragment thereof; an anti-CTLA-4 antibody or antigen-binding fragment thereof; and an OX-40 agonist.

In some embodiments, the immunomodulatory agent is selected from MEDI4736, MPDL3280A, 2.7A4, AMP-714, MDX-1105, nivolumab, pembrolizumab, pidilizumab, BMS936559, MPDL3280A, tremelimumab, ipilimumab, and OX40L FP.

In some embodiments, the immunomodulatory agent is an anti-PD-L1 antibody. In some embodiments, the anti-PD-L1 antibody is MEDI4736.

In some embodiments, the antisense compound targeted to STAT3 does not inhibit STAT1, STAT4, or STAT6. In some embodiments, the antisense compound targeted to STAT3 is an antisense oligonucleotide. In some embodiments, the antisense compound targeted to STAT3 is AZD9150.

In some embodiments, the chemotherapeutic agent is cisplatin, the immunomodulatory agent is MEDI4736, and the antisense compound targeted to STAT3 is AZD9150.

### BRIEF DESCRIPTION OF THE DRAWINGS

FIGS. 1A-1C relate to Example 1. FIG. 1A shows a combined graph of a low dose (5 mg/kg) cisplatin treatment in MC-38 OVA mice. FIG. 1B shows the results of each individual mouse tested. FIG. 1C shows the cisplatin exposure in the mice at different time points after dosing.

FIGS. 2A-2M relate to Example 2. FIGS. 2A-2D show results from treatment of MC-38 OVA mice with: PBS control (FIG. 2A); cisplatin alone, 7 days after tumor implant (FIG. 2B); cisplatin, 3 days after tumor implant and anti-PD-L1 antibody, 7 days after tumor implant (FIG. 2C)); and cisplatin and anti-PD-L1 antibody, concurrently 7 days after tumor implant (FIG. 2D). FIG. 2E shows the body weight of mice measured at various time points after tumor implant and treatment. FIGS. 2F-2M show results from treatment of MC-OVA mice with: PBS control (FIG. 2F); control antisense oligonucleotide (FIG. 2G); cisplatin alone, 7 days after tumor implant (FIG. 2H); cisplatin and anti-PD-L1 antibody, concurrently 7 days after tumor implant (FIG. 2I); STAT3 ASO and anti-PD-L1 antibody, concurrently 7 days after tumor implant (FIG. 2I); STAT3 ASO, 3 days after implant and anti-PD-L1 antibody, 7 days after tumor implant (FIG. 2J); cisplatin, 3 days after tumor implant, STAT3 ASO and anti-PD-L1 antibody, concurrently 7 days after tumor implant (FIG. 2K); STAT3 ASO, 3 days after tumor implant, cisplatin and anti-PD-L1 antibody, concurrently 7 days after tumor implant (FIG. 2L); and cisplatin, anti-PD-L1 antibody, concurrently 7 days after tumor implant (FIG. 2M).

FIG. 3A shows the efficacy of different treatments for tumor-implanted MC38-OVA mice as described in embodiments herein. FIG. 3A shows an average of the results in FIGS. 2A-2D and 2F-2M. FIG. 3B shows the body weight of mice measured at various time points after tumor implant and treatment.

FIG. 4A shows averaged tumor growth results after treatment of MC38 mice with vehicle, STAT3 ASO, anti-PD-L1 antibody, and STAT3 ASO and anti-PD-L1 antibody. FIG. 4B shows the individual results that were averaged and displayed in FIG. 4A. FIG. 4C shows averaged tumor growth results after treatment of MC38 mice with control antibody, anti-PD-L1 antibody, STAT3 ASO alone, or a combination of anti-PD-L1 antibody and STAT3 ASO. FIG. 4D shows the individual results that were averaged and displayed in FIG. 4D.

### DETAILED DESCRIPTION OF THE DISCLOSURE

The present disclosure relates to methods of treating cancer in a patient.

In some embodiments, the nucleic acid molecule, such as an antisense oligonucleotide described herein, can hybridize to a sequence of interest, e.g., a DNA sequence or an RNA sequence. A nucleic acid molecule is “hybridizable” or “hybridized” to another nucleic acid molecule, such as a cDNA, genomic DNA, or RNA, when a single stranded form of the nucleic acid molecule can anneal to the other nucleic acid molecule under the appropriate conditions of temperature and solution ionic strength. In some embodiments, complementary nucleic acid molecules include, but are not limited to, an antisense compound and a nucleic acid target. In some embodiments, complementary nucleic acid molecules include, but are not limited to, a polynucleotide and a target nucleic acid.

Hybridization and washing conditions are known and exemplified in Sambrook et al., Molecular Cloning: A Laboratory Manual, Second Edition, Cold Spring Harbor Laboratory Press, Cold Spring Harbor (1989), particularly Chapter 11 and Table 11.1 therein. The conditions of temperature and ionic strength determine the “stringency” of the hybridization. The stringency of the hybridization conditions can be selected to provide selective formation or maintenance of a desired hybridization product of two complementary nucleic acid polynucleotides, in the presence of other potentially cross-reacting or interfering polynucleotides. Stringent conditions are sequence-dependent; typically, longer complementary sequences specifically hybridize at higher temperatures than shorter complementary sequences. Generally, stringent hybridization conditions are between about 5° C. to about 10° C. lower than the thermal melting point (Tₘ) (i.e., the temperature at which 50% of the sequences hybridize to a substantially complementary sequence) for a specific polynucleotide at a defined ionic strength, concentration of chemical denaturants, pH, and concentration of the hybridization partners. Generally, nucleotide sequences having a higher percentage of G and C bases hybridize under more stringent conditions than nucleotide sequences having a lower percentage of G and C bases. Generally, stringency can be increased by increasing temperature, increasing pH, decreasing ionic strength, and/or increasing the concentration of chemical nucleic acid denaturants (such as formamide, dimethylformamide, dimethylsulfoxide, ethylene glycol, propylene glycol and ethylene carbonate). Stringent hybridization conditions typically include salt concentrations or ionic strength of less than about 1 M, 500 mM, 200 mM, 100 mM or 50 mM; hybridization temperatures above about 20° C., 30° C., 40° C., 60° C. or 80° C.; and chemical denaturant concentrations above about 10%, 20%, 30% 40% or 50%. Because many factors can affect the stringency of hybridization, the combination of parameters may be more significant than the absolute value of any parameter alone.

An exemplary low stringency hybridization condition, for example, corresponding to a Tₘ of 55° C., includes 5× saline-sodium citrate buffer (SSC), 0.1% SDS, 0.25% milk, and no formamide; or 30% formamide, 5×SSC, and 0.5% SDS. An exemplary moderate stringency hybridization condition corresponding to a higher Tₘ of between about 55° C. and about 65° C., includes 40% formamide and 5× or 6×SCC. An exemplary high stringency hybridization condition corresponding to the highest Tₘ of greater than 65° C., includes 50% formamide and 5× or 6×SCC. Further exemplary hybridization conditions include buffered solutions (for example, phosphate, Tris, or HEPES buffered solutions, having between around 20 mM and 200 mM of the buffering component) at pH between around 6.5 to 8.5, and having an ionic strength between about 20 mM and 200 mM, at a temperature between about 15° C. to 40° C. For example, the buffer may include a salt at a concentration of from about 10 mM to about 1 M, from about 20 mM to about 500 mM, from about 30 mM to about 100 mM, from about 40 mM to about 80 mM, or about 50 mM. Exemplary salts include NaCl, KCl, (NH₄)₂SO₄, Na₂SO₄, and CH₃COONH₄.

The term “complementary” is used to describe the relationship between nucleotide bases and/or polynucleotides that are capable of hybridizing to one another, e.g., the nucleotide sequence of such polynucleotides or one or more regions thereof matches the nucleotide sequence of another polynucleotide or one or more regions thereof when the two nucleotide sequences are aligned in opposing directions. Nucleobase matches or complementary nucleobases, as described herein, include the following pairs: adenine (A) and thymine (T), adenine (A) and uracil (U), cytosine (C) and guanine (G), and 5-methyl cytosine (^{m}C) and guanine (G). Complementary polynucleotides and/or nucleic acids need not have nucleobase complementarity at each nucleoside and may include one or more nucleobase mismatches. Accordingly, the present disclosure also includes isolated polynucleotides that are complementary to sequences as disclosed or used herein as well as those substantially similar nucleic acid sequences. The degree to which two polynucleotides have matching nucleobases can be expressed in terms of “percent complementarity” or “percent complementary.” In some embodiments, a polynucleotide has 70%, at least 70%, 75%, at least 75%, 80%, at least 80%, 85%, at least 85%, 90%, at least 90%, 95%, at least 95%, 97%, at least 97%, 98%, at least 98%, 99%, or at least 99% or 100% complementarity with a polynucleotide provided herein. In embodiments wherein two polynucleotides are “fully complementary” or “100% complementary,” such polynucleotides have nucleobase matches at each nucleoside without any nucleobase mismatches.

Unless otherwise modified by the term “intact,” as in “intact antibodies,” the term “antibody” as used herein also includes antibody fragments such as Fab, F(ab′)2, Fv, scFv, Fd, dAb, and other antibody fragments that retain antigen-binding function, for example, the ability to bind, antigens such as CTLA-4, PD1, or PD-L1. Typically, such fragments would comprise an antigen-binding domain.

The term “mAb” refers to monoclonal antibody. Antibodies of the present disclosure can comprise, without limitation, whole native antibodies; bispecific antibodies; chimeric antibodies; Fab, Fab′, single chain V region fragments (scFv); fusion polypeptides; and unconventional antibodies.

As used herein, the terms “sequence similarity” or “% similarity,” and “sequence identity” or “% identity,” refers to the degree of identity or correspondence between nucleic acid sequences or amino acid sequences. In the context of polynucleotides, “sequence similarity” may refer to nucleic acid sequences wherein changes in one or more nucleotide bases results in substitution of one or more amino acids, but do not affect the functional properties of the protein encoded by the polynucleotide. “Sequence similarity” may also refer to modifications of the polynucleotide, such as deletion or insertion of one or more nucleotide bases, that do not substantially affect the functional properties of the resulting transcript. It is therefore understood that the present disclosure encompasses more than the specific exemplary sequences. Methods of making nucleotide base substitutions are known, as are methods of determining the retention of biological activity of the encoded polypeptide.

Moreover, the skilled artisan recognizes that similar polynucleotides encompassed by the present disclosure are also defined by their ability to hybridize, under stringent conditions, with the sequences exemplified herein. Similar polynucleotides of the present disclosure are about 70%, at least about 70%, about 75%, at least about 75%, about 80%, at least about 80%, about 85%, at least about 85%, about 90%, at least about 90%, about 95%, at least about 95%, about 99%, at least about 99%, or about 100% identical to the polynucleotides disclosed herein.

Sequence similarity can be determined by sequence alignment using methods known in the field, such as, for example, BLAST, MUSCLE, Clustal (including ClustalW and ClustalX), and T-Coffee (including variants such as, for example, M-Coffee, R-Coffee, and Expresso).

In some embodiments, only specific portions of two or more polynucleotide or polypeptide sequences are aligned to determine sequence identity. In some embodiments, only specific domains of two or more sequences are aligned to determine sequence similarity. A comparison window can be a segment of at least 10 to over 1000 residues, at least 20 to about 1000 residues, or at least 50 to 500 residues in which the sequences can be aligned and compared. Methods of alignment for determination of sequence identity are well-known and can be performed using publicly available databases such as BLAST. For example, in some embodiments, “percent identity” of two nucleotide sequences is determined using the algorithm of Karlin and Altschul, Proc Nat Acad Sci USA 87:2264-2268 (1990), modified as in Karlin and Altschul, Proc Nat Acad Sci USA 90:5873-5877 (1993). Such algorithms are incorporated into BLAST programs, e.g., BLAST+ or the NBLAST and XBLAST programs described in Altschul et al., J Mol Biol, 215: 403-410 (1990). BLAST protein searches can be performed with programs such as, e.g., the XBLAST program, score=50, wordlength=3 to obtain amino acid sequences homologous to the protein molecules of the disclosure. Where gaps exist between two sequences, Gapped BLAST can be utilized as described in Altschul et al., Nucleic Acids Res 25(17): 3389-3402 (1997). When utilizing BLAST and Gapped BLAST programs, the default parameters of the respective programs (e.g., XBLAST and NBLAST) can be used.

In some embodiments, a polypeptide or polynucleotide has 70%, at least 70%, 75%, at least 75%, 80%, at least 80%, 85%, at least 85%, 90%, at least 90%, 95%, at least 95%, 97%, at least 97%, 98%, at least 98%, 99%, or at least 99% or 100% sequence identity with a reference polypeptide or polynucleotide (or a fragment of the reference polypeptide or polynucleotide) provided herein. In some embodiments, a polypeptide or polynucleotide have about 70%, at least about 70%, about 75%, at least about 75%, about 80%, at least about 80%, about 85%, at least about 85%, about 90%, at least about 90%, about 95%, at least about 95%, about 97%, at least about 97%, about 98%, at least about 98%, about 99%, at least about 99% or about 100% sequence identity with a reference polypeptide or polynucleotide (or a fragment of the reference polypeptide or nucleic acid molecule) provided herein.

As used herein, “dose” means a specified quantity of a pharmaceutical agent provided in a single administration, or in a specified time period. In some embodiments, a dose may be administered in one, two, or more boluses, tablets, or injections. For example, in embodiments wherein subcutaneous administration is desired, the desired dose requires a volume not easily accommodated by a single injection, therefore, two or more injections may be used to achieve the desired dose. In some embodiments, the pharmaceutical agent is administered by infusion over an extended period of time or continuously. Doses may be stated as the amount of pharmaceutical agent per hour, day, week, or month.

As used herein, “parenteral administration” means administration through injection (e.g., bolus injection) or infusion. Parenteral administration includes subcutaneous administration (SC), intravenous administration (IV), intramuscular administration (IM), intraarterial administration (IA), intraperitoneal administration (IP), or intracranial administration (IC), e.g., intrathecal or intracerebroventricular administration.

In some embodiments, the disclosure provides a method of treating cancer in a patient comprising administering to the patient: (a) about 50 mg/m² to about 70 mg/m² chemotherapeutic agent; (b) an immunomodulatory agent; and (c) an antisense compound targeted to STAT3.

As used herein, the term “chemotherapeutic agent” refers to a chemical compound that non-specifically decreases or inhibits the growth, proliferation, and/or survival of a cancerous cell or a cell likely to become cancerous to generate tumorigenic progeny. Such chemical compounds are often directed to intracellular processes necessary for cell growth or division, and are thus particularly effective against cancerous cells, which generally grow and divide rapidly.

Non-limiting examples of chemotherapeutic agents include: oxazaphosphorines such as, e.g., cyclophosphamide and ifosfamide; nitrogen mustards such as, e.g., busulfan, chlorambucil, and melphalan; hydrazines such as, e.g., temozolomide; platinum-based agents, e.g., cisplatin, carboplatin, and oxaliplatin; topoisomerase I inhibitors such as, e.g., irinotecan and topotecan; topoisomerase II inhibitors such as, e.g., etoposide, teniposide, and anthracyclines such as, e.g., doxorubicin, daunorubicin, and idarubicin; vinca alkaloids such as, e.g., vincristine and vinblastine; taxanes such as, e.g., docetaxel and paclitaxel; antifolates such as, e.g., methotrexate and pemetrexed; pyrimidine antagonists such as, e.g., cytarabine, 5-fluorouracil, gemcitabine, and capecitabine; purine analogs such as, e.g., 6-mercaptopurine, azathioprine, and cladribine; purine antagonists such as, e.g., fludarabine; ribonuclease reductase inhibitors such as, e.g., hydroxyurea; antibiotics such as, e.g., bleomycin, actinomycin D, and mitomycin; enzymes such as L-asparaginase; proteasome inhibitors such as bortezomib; tyrosine kinase inhibitors such as imatinib, erlotinib, afatinib; and growth factor inhibitors such as, e.g., gefitinib, cetuximab, and bevacizumab. In some embodiments, the chemotherapeutic agent administered to the patient is a platinum-based agent. In some embodiments, the chemotherapeutic agent is cisplatin.

In general, cisplatin can be used for the treatment of testicular cancer (e.g., metastatic testicular cancer), ovarian cancer (e.g., metastatic ovarian cancer), bladder cancer (e.g., advanced bladder cancer), head and neck cancer, esophageal cancer, small and non-small cell lung cancers, breast cancer, cervical cancer, stomach cancer, prostate cancer, Hodgkin's and non-Hodgkin's lymphomas, neuroblastoma, sarcomas, multiple myeloma, melanoma, and mesothelioma. The typical clinical dosage of cisplatin is about 100 mg/m², which can be administered at once or over several doses in a treatment cycle. Common side effects associated with cisplatin may include nausea and vomiting, leading to weight loss; low blood count; kidney toxicity; ototoxicity; low blood levels of magnesium, calcium, and potassium; peripheral neuropathy; loss of appetite and taste changes; and hair loss.

A reduced dosage of cisplatin can advantageously reduce the side effects associated with cisplatin. In some embodiments, the method comprises administering less than 60 mg/m² cisplatin to the patient. In some embodiments, the method comprises administering about 50 mg/m² to about 70 mg/m² cisplatin to the patient. In some embodiments, the method comprises administering about 50 mg/m² to about 65 mg/m² cisplatin to the patient. In some embodiments, the method comprises administering about 50 mg/m² to about 60 mg/m² cisplatin to the patient. In some embodiments, the method comprises administering about 55 mg/m² to about 60 mg/m² cisplatin to the patient. In some embodiments, the method comprises administering about 50 mg/m², about 51 mg/m², about 52 mg/m², about 53 mg/m², about 54 mg/m², about 55 mg/m², about 56 mg/m², about 57 mg/m², about 58 mg/m², about 59 mg/m², about 60 mg/m², about 61 mg/m², about 62 mg/m², about 63 mg/m², about 64 mg/m², about 65 mg/m², about 66 mg/m², about 67 mg/m², about 68 mg/m², about 69 mg/m², or about 70 mg/m² cisplatin to the patient. In some embodiments, administering the cisplatin in combination with the immunomodulatory agent and the antisense compound targeted to STAT3 allows the cisplatin to be administered at a dose that reduces the side effects compared with administering cisplatin alone.

In some embodiments, the cisplatin is administered to the patient in a single dose. In some embodiments, the cisplatin is administered to the patient in multiple doses, e.g., in 2 doses, 3 doses, 4 doses, 5 doses, 6 doses, 7 doses, 8 doses, 9 doses, 10 doses, or more than 10 doses. In some embodiments, the cisplatin is administered via intraperitoneal administration (IP).

As used herein, “immunomodulatory agent” refers to an agent that enhances an immune response (e.g., antitumor immune response). An immunomodulatory agent can be an antibody or antigen-binding fragment thereof, a protein, a peptide, a small molecule, or combination thereof. In some embodiments, the immunomodulatory agent is an immune checkpoint inhibitor. As used herein, an “immune checkpoint inhibitor” means an agent that inhibits proteins or peptides (i.e., immune checkpoint agents) which are blocking the immune system, e.g., from attacking cancer cells. In some embodiments, the immune checkpoint agent blocking the immune system prevents the production and/or activation of T cells. In some embodiments, the immune checkpoint agent is cytotoxic T lymphocyte associated protein 4 (CTLA-4), programmed cell death protein 1 (PD1), or programmed death ligand 1 (PD-L1). PD-L1 and PD1 form a cell surface-bound ligand-receptor pair that, in healthy individuals, dampen the immune response to prevent an over-reaction of the immune system. In some embodiments, cancer cells hijack the normal PD-L1/PD1 immune checkpoint mechanism by overexpressing the ligand PD-L1, which binds to PD1 on effector CD8 T cells, thereby preventing the T cells from mounting an immune response to the cancer cell and/or tumor. PD-L1 is expressed in a broad range of cancers with high frequently. Tumor PD-L1 overexpression correlates with poor prognosis in a number of cancers (see, e.g., Hamid et al., Expert Opin Biol Ther 13(6):847-861, 2013).

In some embodiments, the immune checkpoint inhibitor inhibits the CTLA-4 pathway or the PD-L1/PD1 pathway. In some embodiments, the immune checkpoint inhibitor is an antibody. In some embodiments, the immune checkpoint inhibitor comprises an antibody that inhibits CTLA-4, PD1, or PD-L1. Immunomodulatory agents, immune checkpoint inhibitors and examples thereof are provided in, e.g., WO 2016/062722.

In some embodiments, the immune checkpoint inhibitor is an anti-PD-L1 antibody or derivative or antigen-binding fragment thereof. In some embodiments, the anti-PD-L1 antibody or derivative or antigen-binding fragment thereof selectively binds a PD-L1 protein or fragment thereof. Examples of anti-PD-L1 antibodies and derivatives and fragments thereof are described in, e.g., WO 01/14556, WO 2007/005874, WO 2009/089149, WO 2011/066389, WO 2012/145493; U.S. Pat. Nos. 8,217,149, 8,779,108; US 2012/0039906, US 2013/0034559, US 2014/0044738, and US 2014/0356353. In some embodiments, the anti-PD-L1 antibody is MEDI4736 (durvalumab), MDPL3280A, 2.7A4, AMP-814, MDX-1105, or atezolizumab (BMS-936559).

In some embodiments, the anti-PD-L1 antibody is MEDI4736. In some embodiments, the anti-PD-L1 antibody comprises an amino acid sequence at least 80%, at least 85%, at least 90%, at least 95%, or at least 99% identical to any of SEQ ID NOs: 3-10. MEDI4736 is an anti-PD-L1 antibody that is selective for a PD-L1 polypeptide and blocks the binding of PD-L1 to the PD-1 and CD80 receptors. MEDI4736 can relieve PD-L1-mediated suppression of human T-cell activation in vitro and can further inhibit tumor growth in a xenograft model via a T-cell dependent mechanism. MEDI4736 is further described in, e.g., U.S. Pat. No. 8,779,108. The fragment crystallizable (Fc) domain of MEDI4736 contains a triple mutation in the constant domain of the IgG1 heavy chain that reduces binding to the complement component C1q and the Fcγ receptors responsible for mediating antibody-dependent cell-mediated cytotoxicity (ADCC).

In some embodiments, MEDI4736 or an antigen-binding fragments thereof comprises a heavy chain and a light chain or a heavy chain variable region and a light chain variable region. In some embodiments, MEDI4736 or an antigen-binding fragment thereof for use comprises a light chain variable region comprising the amino acid sequence of SEQ ID NO: 3 and a heavy chain variable region comprising the amino acid sequence of SEQ ID NO: 4. In some embodiments, MEDI4736 or an antigen-binding fragment thereof comprises a heavy chain variable region and a light chain variable region, wherein the heavy chain variable region comprises the Kabat-defined CDR1, CDR2, and CDR3 sequences of SEQ ID NOS: 5-7, and wherein the light chain variable region comprises the Kabat-defined CDR1, CDR2, and CDR3 sequences of SEQ ID NOS: 8-10. A person of ordinary skill in the art would easily be able to identify Chothia-defined, Abm-defined or other CDR definitions known to those of ordinary skill in the art. In some embodiments, MEDI4736 or an antigen-binding fragment thereof comprises the variable heavy chain and variable light chain CDR sequences of the 2.14H90PT antibody as described in WO 2011/066389.

In some embodiments, the immune checkpoint inhibitor is an anti-PD-1 antibody or derivative or antigen-binding fragment thereof. In some embodiments, the anti-PD-1 antibody selectively binds a PD-1 protein or fragment thereof. In some embodiments, the anti-PD1 antibody is nivolumab, pembrolizumab, pidilizumab, or MPDL3280A.

In some embodiments, the immune checkpoint inhibitor is an anti-CTLA-4 antibody or derivative or antigen-binding fragment thereof. In embodiments, the anti-CTLA-4 antibody selectively binds a CTLA-4 protein or fragment thereof. Examples of anti-CTLA-4 antibodies and derivatives and fragments thereof are described in, e.g., U.S. Pat. Nos. 6,682,736; 7,109,003; 7,123,281; 7,411,057; 7,807,797; 7,824,679; 8,143,379; 8,491,895, and US 2007/0243184. In some embodiments, the anti-CTLA-4 antibody is tremelimumab or ipilimumab. In some embodiments, the anti-CTLA-4 antibody comprises an amino acid sequence at least 80%, at least 85%, at least 90%, at least 95%, or at least 99% identical to any of SEQ ID NOs: 13-20.

In some embodiments, the immunomodulatory agent is an OX40 agonist. OX40 is a tumor necrosis factor receptor (TNFR) found primarily on activated CD4+ and CD8+ T cells, regulatory T cells (Treg), and natural killer (NK) cells. Signaling through OX40 on activated CD4+ and CD8+ T cells leads to enhanced cytokine production, granzyme and perforin release, and expansion of effector and memory T-cell pools. In addition, OX40 signaling on Treg cells inhibits expansion of Tregs, shuts down the induction of Tregs, and blocks Treg-suppressive function. See, e.g., Paterson et al., Mol Immunol 24:1281-1290, 1987; Mallet et al., EMBO J 9:1063-1068, 1990; and Calderhead et al., J Immunol 151:5261-5271, 1993. OX40 is also known in the art as CD134, ACT-4, and ACT-35. Examples of OX40 agonists are described in, e.g., WO 2013/119202; WO 2013/130102; U.S. Pat. Nos. 5,821,332; 6,312,700; 6,156,878; 7,504,101; 7,622,444; and 7,959,925.

In some embodiments, the OX40 agonist is a ligand that specifically binds the OX40 receptor. In some embodiments, the OX40 agonist increases the biological activity of the OX40 receptor. In some embodiments, the biological activity of the OX40 receptor is increased by at least about 10%, 20%, 30%, 40%, 50%, 60%, 70%, 80%, 90%, 95%, 100% or more. In some embodiments, the OX40 agonist is an anti-OX40 antibody. In some embodiments, the OX40 agonist is 9B12, or an antigen-binding fragment or derivative thereof, as described in Weinberg et al., J Immunother 29: 575-585, 2006. In some embodiments, the OX40 agonist is a humanized OX40 antibody, as described by Morris et al., Mol Immunol 44(12):3112-3121, 2007. In some embodiments, the OX40 agonist comprises an amino acid sequence at least 80%, at least 85%, at least 90%, at least 95%, or at least 99% identical to any of SEQ ID NOs: 23, 25, or 26. In some embodiments, the OX40 agonist is an OX40 ligand fusion protein (OX40L FP). In some embodiments, the OX40L FP increases and/or enhances tumor-specific T-cell immunity. In some embodiments, the OX40L FP comprises an amino acid sequence at least 80%, at least 85%, at least 90%, at least 95%, or at least 99% identical to any of SEQ ID NOs: 32, 34, or 36.

In some embodiments, about 0.1 mg/kg to about 20 mg/kg immunomodulatory agent is administered to the patient. In some embodiments, about 1 mg/kg to about 20 mg/kg immunomodulatory agent is administered to the patient. In some embodiments, about 5 mg/kg to about 15 mg/kg immunomodulatory agent is administered to the patient. In some embodiments, about 8 mg/kg to about 12 mg/kg immunomodulatory agent is administered to the patient. In some embodiments, about 10 mg/kg immunomodulatory agent is administered to the patient. In some embodiments, about 0.1 mg/kg, about 0.5 mg/kg, about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 11 mg/kg, about 12 mg/kg, about 13 mg/kg, about 14 mg/kg, about 15 mg/kg, about 16 mg/kg, about 17 mg/kg, about 18 mg/kg, about 19 mg/kg, or about 20 mg/kg immunomodulatory agent is administered to the patient. In some embodiments, the immunomodulatory agent is administered to the patient in a single dose. In some embodiments, the immunomodulatory agent is administered to the patient in multiple doses, e.g., in 2 doses, 3 doses, 4 doses, 5 doses, 6 doses, 7 doses, 8 doses, 9 doses, 10 doses, or more than 10 doses. A person of skill in the art will understand that the particular number of doses and amount in each dose of immunomodulatory agent may be adjusted based on various factors including, e.g., the specific immunomodulatory agent to be administered and the patient's age, disease progression, and/or interactions with the patient's other medications.

In some embodiments, the immunomodulatory agent is MEDI4736. In some embodiments, the method comprises administering a chemotherapeutic agent, about 0.1 mg/kg to about 20 mg/kg MEDI4736, and an antisense compound targeted to STAT3 to the patient. In some embodiments, the method comprises administering a chemotherapeutic agent, about 1 mg/kg to about 20 mg/kg MEDI4736, and an antisense compound targeted to STAT3 to the patient. In some embodiments, the method comprises administering a chemotherapeutic agent, about 3 mg/kg MEDI4736, and an antisense compound targeted to STAT3 to the patient. In some embodiments, the method comprises administering a chemotherapeutic agent, about 10 mg/kg MEDI4736, and an antisense compound targeted to STAT3 to the patient. In some embodiments, the method comprises administering a chemotherapeutic agent, about 20 mg/kg MEDI4736, and an antisense compound targeted to STAT3 to the patient. In some embodiments, the immunomodulatory agent is administered intraperitoneally. In some embodiments, the chemotherapeutic agent and the immunomodulatory agent are both administered intraperitoneally. In some embodiments, the chemotherapeutic agent and the immunomodulatory agent are co-administered intraperitoneally (i.e., in the same dosage form). In some embodiments, the chemotherapeutic agent and the immunomodulatory agent are separately administered intraperitoneally (i.e., each agent in a separate dosage form).

As used herein, the term “antisense compound” means an oligomeric compound that is capable of undergoing hybridization to a target nucleic acid, e.g., through hydrogen bonding. Examples of antisense compounds include single-stranded and double-stranded compounds, such as, e.g., antisense oligonucleotides (ASO), small interfering RNAs (siRNA), short hairpin RNAs (shRNA), small nucleolar RNAs (snoRNA), microRNAs (miRNA), and meroduplexes (mdRNA), and satellite repeat sequences.

An “antisense oligonucleotide” or “ASO” refers to a polynucleotide comprising a sequence that is complementary to a target nucleic acid or region or segment thereof. In some embodiments, an ASO is specifically hybridizable to a target nucleic acid or region or segment thereof. In some embodiments, ASOs are capable of influencing RNA processing and/or modulating protein expression. In general, an ASO is a single-stranded oligonucleotide that binds to single-stranded RNA to inactivate the RNA. In some embodiments, an ASO binds to messenger RNA (mRNA) for a gene, thereby inactivating the gene. In some embodiments, an ASO binds to a transcription initiation site, a translation initiation site, 5′-untranslated sequence, 3′-untranslated sequence, coding sequence, a pre-mRNA sequence, an mRNA splice site, and/or an intron/exon junction of an mRNA encoding a gene, thereby inactivating the gene. In some embodiments, the ASO includes DNA, RNA, or combination thereof. ASOs are further described in, e.g., Goodchild, Methods Mol Biol 764:1-15, 2011; Smith et al., Ann Rev Pharmacol Toxicol 59:605-630, 2019; and Stein et al., Mol Ther 25(5):1069-1075, 2017.

As described herein, Signal Transducer and Activator of Transcription 3 (STAT3) is a transcription factor and master regulator of immune suppression known to promote oncogenesis. In some embodiments, an antisense compound targeted to STAT3 is an oligomeric compound capable of specifically hybridizing to the STAT3 target nucleic acid. In some embodiments, the antisense compound targeted to STAT3 inhibits the transcription and/or translation of STAT3. Antisense compounds and antisense oligonucleotides, e.g., targeting STAT3, are provided in, e.g., WO 2016/062722.

While STAT3 regulates immune suppression and is involved in oncogenesis, other members of the STAT family, which may be similar in structure and/or sequence to STAT3, perform different functions. For example, STAT1 enhances inflammation and innate and adaptive immunity, triggering in most instances anti-proliferative and pro-apoptotic responses in tumor cells. STAT4 has been shown to be important in anti-tumor T_{H}1 responses, and STAT6 was shown to play a role in interleukin-4-mediated growth inhibition and induction of apoptosis. See, e.g., Gooch et al., Neoplasia 4(4):324-331, 2002; Yu et al., Nat Rev Cancer 9(11):798-809, 2009; and Kamran et al., Biomed Res Int 2013:421821, 2013. In some embodiments, the antisense compound targeted to STAT3 does not hybridize to STAT1, STAT4, or STAT6. In some embodiments, the antisense compound targeted to STAT3 does not inhibit STAT1, STAT4, or STAT6.

In some embodiments, the STAT3 target nucleic acid includes any nucleic acid encoding STAT3. In some embodiments, the STAT3 target nucleic acid includes a DNA sequence encoding STAT3, an RNA sequence transcribed from DNA encoding STAT3 (including genomic DNA comprising introns and exons), and an mRNA sequence encoding STAT3. Exemplary antisense compounds targeted to STAT3, including antisense oligonucleotides, are described in, e.g., WO 2000/061602; WO 2005/083124; WO 2012/135736; WO 2014/070868; WO 2008/109494; and US 2010/0298409. In some embodiments, the antisense compound targeted to STAT3 is an antisense oligonucleotide. In some embodiments, the antisense oligonucleotide comprises a nucleotide sequence at least 50%, at least 55%, at least 60%, at least 65%, at least 70%, at least 75%, at least 80%, at least 85%, at least 90%, at least 95%, or about 100% complementary to a portion or all of a nucleic acid encoding STAT3 (SEQ ID NO: 1). In some embodiments, the antisense oligonucleotide comprises a nucleotide sequence at least 90%, at least 91%, at least 92%, at least 93%, at least 94%, at least 95%, at least 96%, at least 97%, at least 98%, at least 99%, or about 100% complementary to a portion or all of a nucleic acid encoding STAT3 (SEQ ID NO: 1).

In some embodiments, the antisense compound targeted to STAT3 is AZD9150. The nucleotide sequence of AZD9150 is provided in SEQ ID NO: 2. In some embodiments, the antisense compound targeted to STAT3 comprises a nucleotide sequence at least 80%, at least 85%, at least 90%, at least 95%, or at least 99% identical to SEQ ID NO: 2.

In some embodiments, about 100 mg to about 500 mg antisense compound targeted to STAT3 is administered to the patient. In some embodiments, about 200 mg to about 400 mg antisense compound targeted to STAT3 is administered to the patient. In some embodiments, about 100 mg, about 125 mg, about 150 mg, about 175 mg, about 200 mg, about 225 mg, about 250 mg, about 275 mg, about 300 mg, about 325 mg, about 350 mg, about 375 mg about 400 mg, about 425 mg, about 450 mg, about 475 mg, or about 500 mg antisense compound targeted to STAT3 is administered to the patient. In some embodiments, the antisense compound targeted to STAT3 is administered to the patient in a single dose. In some embodiments, the antisense compound targeted to STAT3 is administered to the patient in multiple doses, e.g., in 2 doses, 3 doses, 4 doses, 5 doses, 6 doses, 7 doses, 8 doses, 9 doses, 10 doses, or more than 10 doses. A person of skill in the art will understand that, in a similar manner as the immunomodulatory agent described herein, the particular number of doses and amount in each dose of the antisense compound targeted to STAT3 may be adjusted based on various factors including, e.g., the specific antisense compound to be administered and the patient's age, disease progression, and/or interactions with the patient's other medications. In some embodiments, the antisense compound targeted to STAT3 is administered subcutaneously. In some embodiments, the antisense compound targeted to STAT3 is administered subcutaneously, and the chemotherapeutic agent and the immunomodulatory agent are administered intraperitoneally as described herein.

In some embodiments, the antisense compound targeted to STAT3 is AZD9150. In some embodiments, the method comprises administering a chemotherapeutic agent, an immunomodulatory agent, and about 100 mg to about 500 mg AZD9150 to the patient. In some embodiments, the method comprises administering a chemotherapeutic agent, an immunomodulatory agent, and about 200 mg to about 400 mg AZD9150 to the patient. In some embodiments, the method comprises administering a chemotherapeutic agent, an immunomodulatory agent, and about 300 mg AZD9150 to the patient.

In some embodiments, the chemotherapeutic agent is cisplatin, the immunomodulatory agent is MEDI4736, and the antisense compound targeted to STAT3 is AZD9150. In some embodiments, the disclosure provides a method of treating cancer in a patient, comprising administering to the patient (a) about 50 mg/m² to about 60 mg/m² cisplatin; (b) about 1 mg/kg to about 200 mg/kg MEDI4736; and (c) about 200 mg to about 400 mg AZD9150. In some embodiments, the method comprises administering about 60 mg/m² cisplatin, about 10 mg/kg MEDI4736, and about 300 mg AZD9150 to the patient. In some embodiments, administration of the combination of the chemotherapeutic agent, immunomodulatory agent, and antisense compound targeted to STAT3 as described herein results in an additive and/or synergistic effect. As used herein, the term “synergistic” refers to a combination of therapies (e.g., a combination of cisplatin, MEDI4736 or an antigen-binding fragment thereof, and AZD9150 as described herein), which is more effective than the additive effects of the single therapies.

A synergistic effect of a combination of therapies (e.g., a combination of cisplatin, MEDI4736 or an antigen-binding fragment thereof, and AZD9150 as described herein) may permit the use of lower dosages of one or more of the therapeutic agents and/or less frequent administration of said therapeutic agents to a patient with cancer. The ability to utilize lower dosages of therapeutic agents and/or to administer said therapies less frequently reduces the toxicity associated with the administration of said therapies to a subject without reducing the efficacy of said therapies in the treatment of a cancer. In addition, a synergistic effect can result in improved efficacy of therapeutic agents in the management, treatment, or amelioration of a cancer. The synergistic effect of a combination of therapeutic agents can avoid or reduce adverse or unwanted side effects associated with the use of either single therapy. The synergistic effect of a combination of therapeutic agents may also manifest itself as a reduction in tumor mass (or tumor regression). The synergistic effect of a combination of therapeutic agents may also manifest itself as a sustained reduction in tumor growth rate.

In some embodiments, the method comprises administering the chemotherapeutic agent, the immunomodulatory agent, and the antisense compound targeted to STAT3 to the patient in one or more treatment cycles. A “treatment cycle,” in the context of cancer treatment, refers to a period of treatment (e.g., administration of one or more agents) followed by a period of rest (no treatment) that is repeated on a regular schedule. For example, treatment can be given for one week, followed by three weeks of rest is one treatment cycle. In some embodiments, a treatment cycle is about 1 day to about 3 months. In some embodiments, a treatment cycle is about 5 days to about 1 month. In some embodiments, a treatment cycle is about 1 week to about 3 weeks. In some embodiments, a treatment cycle is about 1 day, about 3 days, about 1 week, about 10 days, about 2 weeks, about 3 weeks, about 4 weeks, about 2 months, about 3 months, or about 100 days. In some embodiments, the period of rest in a treatment cycle is about 1 day to about 1 month. In some embodiments, the period of rest in a treatment cycle is about 1 day, about 3 days, about 5 days, about 1 week, about 2 weeks, about 3 weeks, or about 4 weeks.

A “treatment course” comprises multiple treatment cycles, which can be repeated on a regular schedule, or adjusted as a tapered schedule as the patient's disease progression is monitored. For example, a patient's treatment cycles can have longer periods of treatment and/or shorter periods of rest at the beginning of a treatment course (e.g., when the patient is first diagnosed), and as the cancer enters remission, the rest period lengthens, thereby increasing the length of one treatment cycle. The period of time for treatment and rest in a treatment cycle, the number of treatment cycles, and the length of time for the treatment course can be determined and adjusted throughout the treatment course by the skilled artisan based on the patient's disease progression, treatment tolerance, and prognosis. In some embodiments, the method comprises 1 to 10 treatment cycles. In some embodiments, the method comprises 2 to 8 treatment cycles.

In a treatment cycle, one or more therapeutic agents (e.g., a chemotherapeutic agent, an immunomodulatory agent, and/or an antisense compound) can be administered concurrently or at different times during the treatment cycle. In some embodiments, the chemotherapeutic agent, the immunomodulatory agent, and the antisense compound targeted to STAT3 are administered to the patient concurrently in a treatment cycle. In some embodiments, the chemotherapeutic agent is cisplatin, the immunomodulatory agent is MEDI4736, and the antisense compound targeted to STAT3 is AZD9150 as described herein.

In some embodiments, the chemotherapeutic agent is administered to the patient before the immunomodulatory agent and the antisense compound targeted to STAT3. In some embodiments, the chemotherapeutic agent is administered to the patient about 12 hours, about 1 day, about 2 days, about 3 days, about 4 days, about 5 days, about 6 days, about 1 week, about 10 days, or about 2 weeks before the immunomodulatory agent and the antisense compound targeted to STAT3.

In some embodiments, after administering the chemotherapeutic agent, the immunomodulatory agent and the antisense compound targeted to STAT3 are administered concurrently. In some embodiments, the immunomodulatory agent and the antisense compound targeted to STAT3 are administered at different time points, e.g., about 10 minutes apart, about 30 minutes apart, 1 hour apart, about 2 hours apart, about 4 hours apart, about 8 hours apart, about 12 hours apart, about 1 day apart, about 2 days apart, about 3 days apart, about 4 days apart, about 5 days apart, about 6 days apart, about 1 week apart, about 10 days apart, or about 2 weeks apart from one another, in either order (e.g., administration of the immunomodulatory agent followed by the antisense compound targeted to STAT3, or administration of the antisense compound targeted to STAT3, followed by administration of the immunomodulatory agent). In some embodiments, the chemotherapeutic agent is administered first, followed by the immunomodulatory agent, followed by the antisense compound targeted to STAT3. In some embodiments, the chemotherapeutic agent is administered first, followed by the antisense compound targeted to STAT3, followed by the immunomodulatory agent.

In some embodiments, the chemotherapeutic agent and the immunomodulatory agent are administered to the patient before the antisense compound targeted to STAT3. In some embodiments, the chemotherapeutic agent and the immunomodulatory agent are administered concurrently before administration of the antisense compound targeted to STAT3. In some embodiments, the chemotherapeutic agent and the immunomodulatory agent are administered to the patient about 12 hours, about 1 day, about 2 days, about 3 days, about 4 days, about 5 days, about 6 days, about 1 week, about 10 days, or about 2 weeks before the antisense compound targeted to STAT3. In some embodiments, the chemotherapeutic agent and the immunomodulatory agent are administered at different time points before administration of the antisense compound targeted to STAT3, as described herein.

In some embodiments, the chemotherapeutic agent and the antisense compound targeted to STAT3 are administered to the patient before the immunomodulatory agent. In some embodiments, the chemotherapeutic agent and the antisense compound targeted to STAT3 are administered concurrently before administration of the immunomodulatory agent. In some embodiments, the chemotherapeutic agent and the antisense compound targeted to STAT3 are administered to the patient about 12 hours, about 1 day, about 2 days, about 3 days, about 4 days, about 5 days, about 6 days, about 1 week, about 10 days, or about 2 weeks before the immunomodulatory agent. In some embodiments, the chemotherapeutic agent and the antisense compound targeted to STAT3 are administered at different time points before administration of the immunomodulatory agent, as described herein.

In some embodiments, a treatment cycle comprises administering one or more doses of the chemotherapeutic agent, the immunomodulatory agent, and/or the antisense compound targeted to STAT3. In some embodiments, the chemotherapeutic agent is administered in 1, 2, 3, 4, 5, 6, 7, 8, 9, 10, or more than 10 doses in a treatment cycle. In some embodiments, the immunomodulatory agent is administered in 1, 2, 3, 4, 5, 6, 7, 8, 9, 10, 11, 12, 13, 14, 15, 16, 17, 18, 19, 20, or more than 20 doses in a treatment cycle. In some embodiments, the antisense compound targeted to STAT3 is administered in 1, 2, 3, 4, 5, 6, 7, 8, 9, 10, 11, 12, 13, 14, 15, 16, 17, 18, 19, 20, or more than 20 doses in a treatment cycle. In embodiments wherein multiple doses are administered, the multiple doses can be administered multiple times per day and/or multiple times per week. For example, the multiple doses can be administered about 1, 2, 3, 4, 5, or more than 5 days per week, and/or about 1, 2, 3, 4, 5, or more than 5 days per week.

In embodiments wherein at least two agents (e.g., the chemotherapeutic agent and the immunomodulatory agent, the chemotherapeutic agent and the antisense compound targeted to STAT3, the immunomodulatory agent and the antisense compound targeted to STAT3, or all of the above) are administered concurrently, and at least one of the agents is administered in multiple doses, it should be understood that at least one of the multiple doses of such agent(s) is administered concurrently with the other agent(s).

In some embodiments, fewer doses of the chemotherapeutic agent are administered to the patient than the immunostimulatory agent and the antisense compound targeted to STAT3 on a treatment cycle. In some embodiments, about 1 dose of the chemotherapeutic agent, about 1 to about 10 doses of the immunomodulatory agent, and about 1 to about 20 doses of the antisense compound targeted to STAT3 are administered to the patient in a treatment cycle. In some embodiments, about 1 dose of the chemotherapeutic agent, about 2 to about 5 doses of the immunomodulatory agent, and about 5 to about 20 doses of the antisense compound targeted to STAT3 are administered to the patient in a treatment cycle. In some embodiments, about 1 dose of the chemotherapeutic agent, about 4 doses of the immunomodulatory agent, and about 15 doses of the antisense compound targeted to STAT3 are administered to the patient in a treatment cycle.

A non-limiting example of a treatment cycle includes: a single dose of about 50 mg/m² to about 70 mg/m² of chemotherapeutic agent, e.g., cisplatin; about 1 mg/kg to about 20 mg/kg of immunomodulatory agent, e.g., MEDI4736, administered 2 times per week for 2 weeks; and about 200 mg to about 400 mg of antisense compound targeted to STAT3, e.g., AZD9150, administered 5 times per week for 3 weeks. In some embodiments, the chemotherapeutic agent is administered, e.g., about 12 hours to about 2 weeks before the immunomodulatory agent and/or the antisense compound targeted to STAT3, as described herein.

In some embodiments, the method provided herein, e.g., administration of all three of a chemotherapeutic agent, an immunomodulatory agent, and an antisense compound targeted to STAT3, advantageously minimizes direct T cell killing with chemotherapy, enhances antigen presentation, and/or promotes T cell activation, and thereby provides a safer and more effective treatment to the patient, compared with a method that administers only one or only two of agents. In some embodiments, the method provided herein results in an increase in CD11b+/Ly6C+ dendritic cells compared to administration of the immunomodulatory agent alone, administration of the antisense compound targeted to STAT3 alone, administration of the chemotherapeutic agent alone, or administration of a combination of any two of the agents, e.g., a chemotherapeutic agent and an immunomodulatory agent and or a chemotherapeutic agent and an antisense compound targeted to STAT3. In some embodiments, CD11b+/Ly6C+ cells suppress IL-17 production. In some embodiment, an increase in CD11b+/Ly6C+ cells inhibit tumor growth.

In some embodiments, the method provided herein results in enhanced CD4 T cell functionality compared with a method administering only one or only two of the three agents. In some embodiments, the method provided herein results in a 1.1-fold, 1.2-fold, 1.3-fold, 1.4-fold, 1.5-fold, 1.6-fold, 1.7-fold, 1.8-fold, 1.9-fold, 2-fold, or more than 2-fold increase in interferon-γ (IFNγ) levels in the patient compared with a method administering only one or only two of the three agents. In some embodiments, the method provided herein results in a 1.1-fold, 1.2-fold, 1.3-fold, 1.4-fold, 1.5-fold, 1.6-fold, 1.7-fold, 1.8-fold, 1.9-fold, 2-fold, or more than 2-fold increase in interleukin-2 (IL-2) levels in the patient compared with a method administering only one or only two of the three agents.

In some embodiments, the method provided herein results in enhanced natural killer (NK) cell functionality compared with a method administering only one or only two of the agents. In some embodiments, the method provided herein results in a 1.1-fold, 1.2-fold, 1.3-fold, 1.4-fold, 1.5-fold, 1.6-fold, 1.7-fold, 1.8-fold, 1.9-fold, 2-fold, or more than 2-fold increase in granzyme B+ levels in the patient compared with a method administering only one or only two of the three agents. In some embodiments, the method provided herein results in a 1-fold, 1.3-fold, 1.5-fold, 1.8-fold, 2-fold, 2.3-fold, 2.5-fold, 2.8-fold, 3-fold, 3.3-fold, 3.5-fold, 3.8-fold, 4-fold, 4.3-fold, 4.5-fold, 4.8-fold, 5-fold, 10-fold, or more than 10-fold increase in tumor necrosis factor alpha (TNFα) levels in the patient compared with a method administering only one or only two of the three agents.

In some embodiments, the patient has cancer. In some embodiments, the cancer is breast cancer, including triple negative breast cancer; ovarian cancer, including serous ovarian cancer; renal carcinoma; lung cancer, including non-small cell lung cancer (NSCLC); pancreatic cancer; colorectal cancer; hepatocellular carcinoma (HCC); head and neck cancer, including squamous cell carcinoma (HNSCC); or lymphoma, including diffuse large B-cell carcinoma (DLBCL) and Hodgkin's lymphoma. In some embodiments, the cancer is non-small cell lung cancer (NSCLC), squamous cell carcinoma, adenocarcinoma, large cell carcinoma, adenosquamous carcinoma, or sarcomatoid carcinoma. In some embodiments, the cancer is head and neck squamous cell carcinoma (HNSCC). In some embodiments, the cancer is diffuse large B-cell carcinoma (DLBCL).

In some embodiments, the patient has a PD-L1 positive cancer. A “PD-L1 positive” cancer means that cells in a cancer sample exhibit immunohistochemistry staining for PD-L1. The level of positivity that is biological or clinically significant can vary, based on tumor type and/or the immune status of the tumor environment. In some embodiments, the patient comprises cancer cells expressing PD-L1. In some embodiments, at least 20%, 25%, 30%, 35%, 40%, 45%, 50%, 55%, 60%, 65%, 70%, 75%, 80%, or more of the cells in the tumor of the patient are PD-L1 positive when assessed using immunochemistry.

In some embodiments, the method provided herein results in an increase in progression-free survival and/or overall survival as compared to administration of the immunomodulatory agent alone, administration of the antisense compound targeted to STAT3 alone, or administration of the chemotherapeutic agent alone. As used herein, “progression-free survival” means the length of time during and after the treatment of a disease, such as cancer, that a patient is living with the disease but the disease does not get worse. Progression-free survival can typically be determined by the skilled artisan, e.g., as an average from an appropriately sized clinical trial. As used herein, “overall survival” means the length of time from the start of treatment for a disease, such as cancer, that patients diagnosed with the disease are still alive. Overall survival can typically be determined as an average from an appropriately sized clinical trial.

In some embodiments, the methods provided herein decrease and/or inhibit cancer tumor growth. Reduction in tumor growth can be measured, e.g., by comparison to the growth of patient's tumor at baseline, against an expected tumor growth, against an expected tumor growth based on a large patient population, or against the tumor growth of a control population.

In some embodiments, a tumor response is measured to determine efficacy of the treatment, e.g., the method provided herein. In some embodiments, a tumor response is measured using the Immune-related Response Criteria (irRc), e.g., as described in Wolchok et al., Cancer Therapy 15(23):7412-7420, 2009. In some embodiments, a tumor response is measured using the Response Evaluation Critera in Solid Tumors (RECIST), e.g., as described in Eisenhauer et al., Eur J Cancer 45:288-247, 2009. In some embodiments, a tumor response is detectable at week 4 or thereafter, e.g., at week 7, week 10, week 13, week 20, week 25, week 30, week 35, week 40, week 41, week 45, week 50, or week 52.

In certain embodiments, a patient achieves disease control (DC). Disease control can be a complete response (CR), partial response (PR), or stable disease (SD). A “complete response” (CR) refers to the disappearance of all lesions, whether measurable or not, and no new lesions. Confirmation can be obtained using a repeat, consecutive assessment no less than four weeks from the date of first documentation. New, non-measurable lesions preclude CR. A “partial response” (PR) refers to a decrease in tumor burden of greater than 30% relative to baseline. Confirmation can be obtained using a consecutive repeat assessment at least 4 weeks from the date of first documentation. “Stable disease” (SD) indicates a decrease in tumor burden of less than about 30% relative to baseline cannot be established and a 20% or greater increase compared to nadir cannot be established.

In some embodiments, the disclosure provides a pharmaceutical composition comprising (a) a chemotherapeutic agent; and (b) an immunomodulatory agent, wherein the chemotherapeutic agent and the immunomodulatory agent are in the pharmaceutical composition at a weight ratio of about 1:1 to about 1:4. In some embodiments, the chemotherapeutic agent and the immunomodulatory agent are in the pharmaceutical composition at a weight ratio of about 1:2. Chemotherapeutic agents and immunomodulatory agents are described herein. In some embodiments, the chemotherapeutic agent is cisplatin. In some embodiments, the immunomodulatory agent is MEDI4736 or a derivative or antigen-binding fragment thereof. In some embodiments, the pharmaceutical composition further comprises a pharmaceutically acceptable excipient, e.g., tonicity adjusting agent, preservative, solubilizing agent, complexing agent, dispersing agent, buffering agent, or combination thereof. In some embodiments, the pharmaceutical composition is suitable for administration to a patient. In some embodiments, the pharmaceutical composition is suitable for intraperitoneal administration to a patient.

In some embodiments, the disclosure further provides a first pharmaceutical composition described herein, comprising (a) a chemotherapeutic agent; and (b) an immunomodulatory agent, wherein the chemotherapeutic agent and the immunomodulatory agent are in the pharmaceutical composition at a weight ratio of about 1:1 to about 1:4; and a second pharmaceutical composition comprising an antisense compound targeted to STAT3. Antisense compounds targeted to STAT3 are described herein. In some embodiments, the second pharmaceutical composition further comprises a pharmaceutically acceptable excipient, e.g., as described herein. In some embodiments, the second pharmaceutical composition is suitable for subcutaneous administration to a patient. In some embodiments, the chemotherapeutic agent is cisplatin. In some embodiments, the immunomodulatory agent is MEDI4736 or a derivative or antigen-binding fragment thereof. In some embodiments, the antisense compound targeted to STAT3 is AZD9150. In some embodiments, the first and second pharmaceutical compositions are provided to a patient in need of treatment. In some embodiments, the patient has cancer. Various types of cancers are described herein.

In some embodiments, the disclosure further provides a kit for treating cancer, comprising: (a) a chemotherapeutic agent; (b) an immunomodulatory agent; and (c) an antisense compound targeted to STAT3. Chemotherapeutic agents, immunomodulatory agents, and antisense compounds targeted to STAT3 are described herein. In some embodiments, the chemotherapeutic agent is cisplatin. In some embodiments, the immunomodulatory agent is MEDI4736 or a derivative or antigen-binding fragment thereof. In some embodiments, the antisense compound targeted to STAT3 is AZD9150.

In some embodiments, the kit comprises a sterile container which contains one or more therapeutic compositions; such containers can be boxes, ampoules, bottles, vials, tubes, bags, pouches, blister-packs, or other suitable container forms known in the art. Such containers can be made of plastic, glass, laminated paper, metal foil, or other materials suitable for holding medicaments.

In some embodiments, the kit further comprises instructions for administering the chemotherapeutic agent (e.g., cisplatin), the immunomodulatory agent (e.g., MEDI4736), and the antisense compound targeted to STAT3 (e.g., AZD9150) to a subject having a cancer. In some embodiments, the instructions include at least one of the following: description of the therapeutic agent(s); dosage schedule and administration for treatment or prevention of cancer or symptoms thereof; precautions; warnings; indications; counter-indications; over dosage information; adverse reactions; animal pharmacology; clinical studies; and/or references. The instructions can be printed directly on the container (when present), or as a label applied to the container, or as a separate sheet, pamphlet, card, or folder supplied in or with the container.

All references cited herein, including patents, patent applications, papers, textbooks and the like, and the references cited therein, to the extent that they are not already, are hereby incorporated herein by reference in their entirety.

### EXAMPLES

### Example 1

### Identification of a Low Dose Cisplatin Treatment

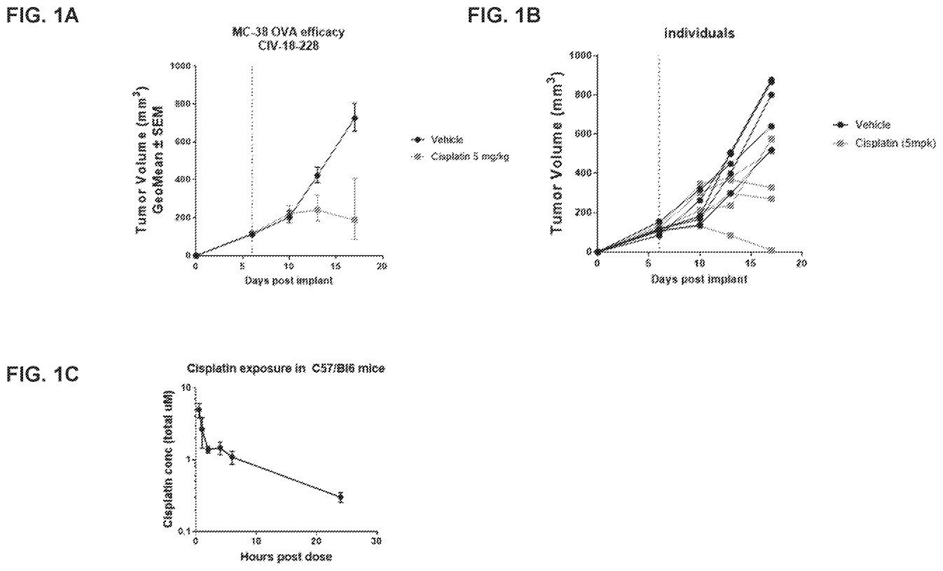

A low dose treatment of cisplatin, 5 mg/kg (equivalent to approximately 60 mg/m² human dose) was tested for anti-tumor activity in MC-38 OVA mice. Results in FIGS. 1A-1C indicate that the dose produces most tumor growth inhibition.

### Example 2

### Anti-Tumor Activity of Therapeutic Agent Combinations

Various combinations and dosing schedules of cisplatin, an anti-PD-L1 antibody, and a STAT3 antisense oligonucleotide (ASO) were tested for efficacy in MC-38 OVA mouse models, and also to evaluate activity of cross-priming dendritic cells from tumor-draining lymph nodes in co-culture assays with OTI/OTII T cells. The tested therapeutic agent combinations are shown in Table 1.

TABLE 1

| ID | Therapeutic Agents | Amount | Route | Dosing Frequency | 1st Post-Implant Dose |
|---|---|---|---|---|---|
| 1 | Control ASO | 50 mg/kg | SC | QD × 5/wk | |
| 2 | Cisplatin | 5 mg/kg | IP | Single dose | Day 7 |
| 3 | Cisplatin | 5 mg/kg | IP | Single dose | Day 7 |
| | PD-L1 Ab | 10 mg/kg | IP | Biweekly | Day 7 |
| 4 | Cisplatin | 5 mg/kg | IP | Single dose | Day 3 |
| | PD-L1 Ab | 10 mg/kg | IP | Biweekly | Day 7 |
| 5 | STAT3 ASO | 50 mg/kg | SC | QD × 5/wk × 3 wk | Day 7 |
| | PD-L1 Ab | 10 mg/kg | IP | 2×/wk × 2 wk | Day 7 |
| 6 | STAT3 ASO | 50 mg/kg | SC | QD × 5/wk × 3 wk | Day 3 |
| | PD-L1 Ab | 10 mg/kg | IP | 2×/wk × 2 wk | Day 7 |
| 7 | STAT3 ASO | 50 mg/kg | SC | QD × 5/wk × 3 wk | Day 7 |
| | PD-L1 Ab | 10 mg/kg | IP | 2×/wk × 2 wk | Day 7 |
| | Cisplatin | 5 mg/kg | IP | Single dose | Day 7 |
| 8 | STAT3 ASO | 50 mg/kg | SC | QD × 5/wk × 3 wk | Day 3 |
| | PD-L1 Ab | 10 mg/kg | IP | 2×/wk × 2 wk | Day 7 |
| | Cisplatin | 5 mg/kg | IP | Single dose | Day 7 |
| 9 | STAT3 ASO | 50 mg/kg | SC | QD × 5/wk × 3 wk | Day 7 |
| | PD-L1 Ab | 10 mg/kg | IP | 2×/wk × 2 wk | Day 7 |
| | Cisplatin | 5 mg/kg | IP | Single dose | Day 3 |

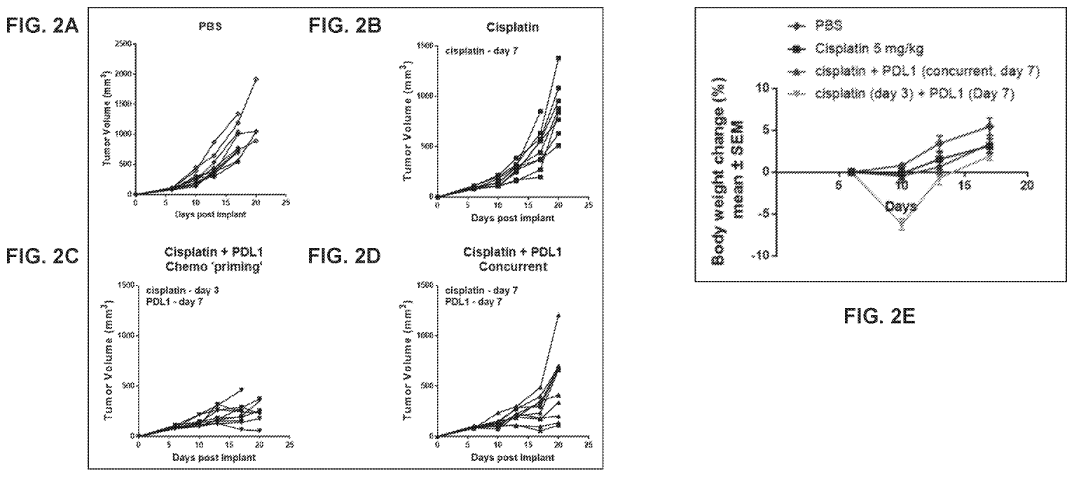

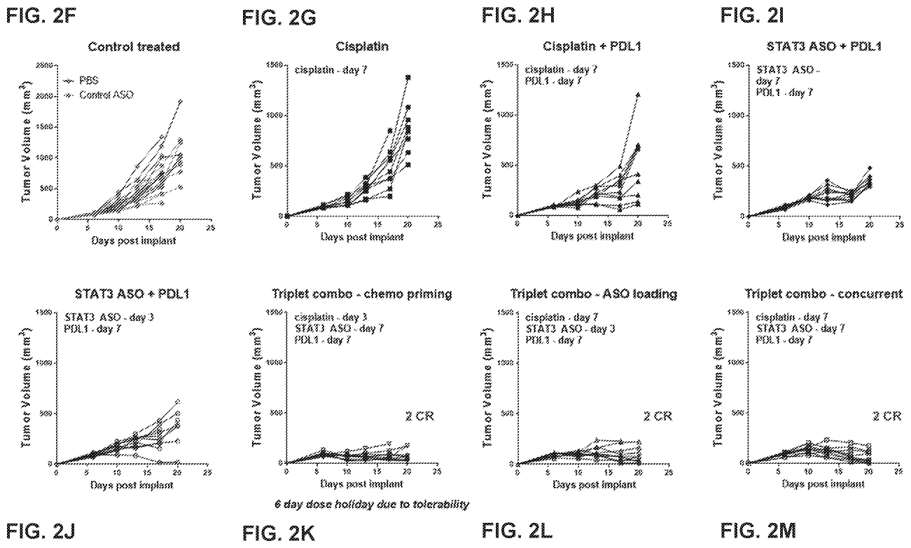

Results in FIGS. 2A-2D and 2F-2M show tumor growth after administering the agents according to Table 1. The combination of cisplatin and anti-PD-L1 antibody (FIGS. 2C and 2D) had similar anti-tumor efficacy and improved over PBS control (FIG. 2A) or cisplatin alone (FIG. 2B). FIGS. 2F-2M further demonstrate that the combinations of cisplatin, anti-PD-L1 antibody, and STAT3 ASO (FIGS. 2K-2M) had the best performance in driving tumor regression compared with PBS control or control ASO (FIG. 2F), cisplatin alone (FIG. 2G), cisplatin in combination with either the anti-PD-L1 antibody (FIG. 2H) or the STAT3 ASO (FIG. 2I), or the anti-PD-L1 antibody in combination with the STAT3 ASO (FIGS. 2I and 2J). FIG. 2E shows the body weight of mice treated with the agents indicated in FIGS. 2A-2D.

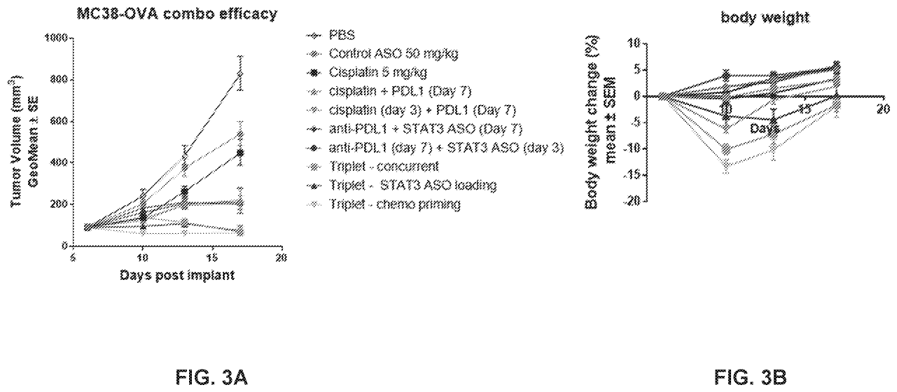

FIG. 3A shows a combined graph of the results in FIGS. 2A-2B. All three of the tested “triple combinations” (i.e., combination of cisplatin, anti-PD-L1 antibody, and STAT3 ASO) resulted in tumor stasis and greater anti-tumor efficacy compared with combinations of only two of the three agents. FIG. 3B shows a combined graph of the body weight changes associated with each of the treatments.

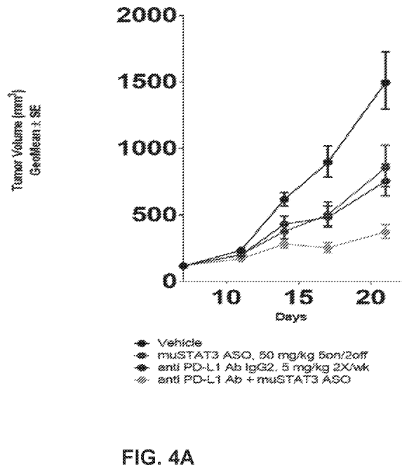

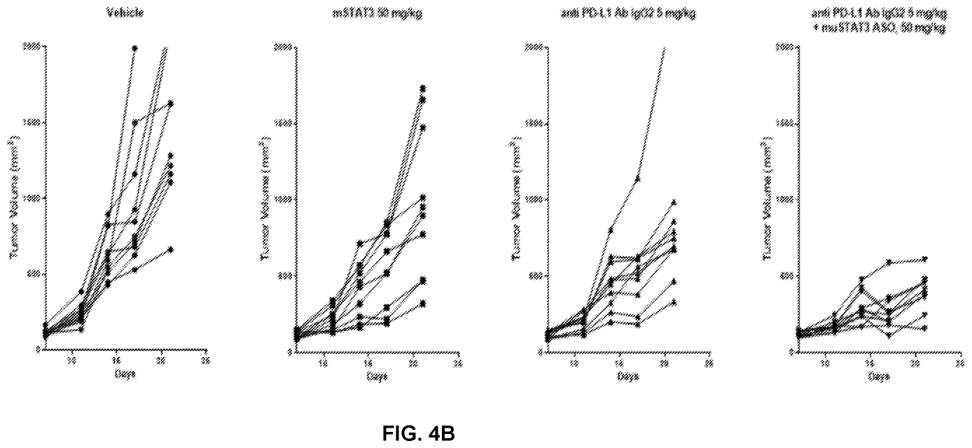

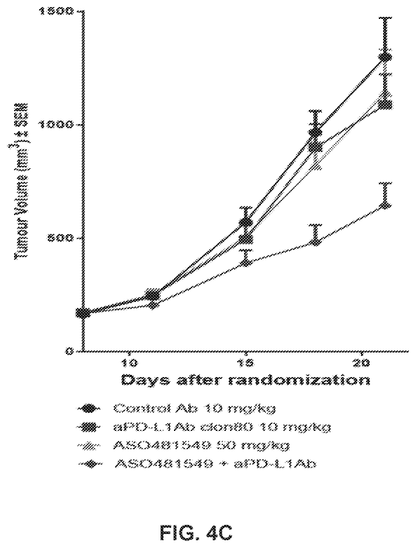

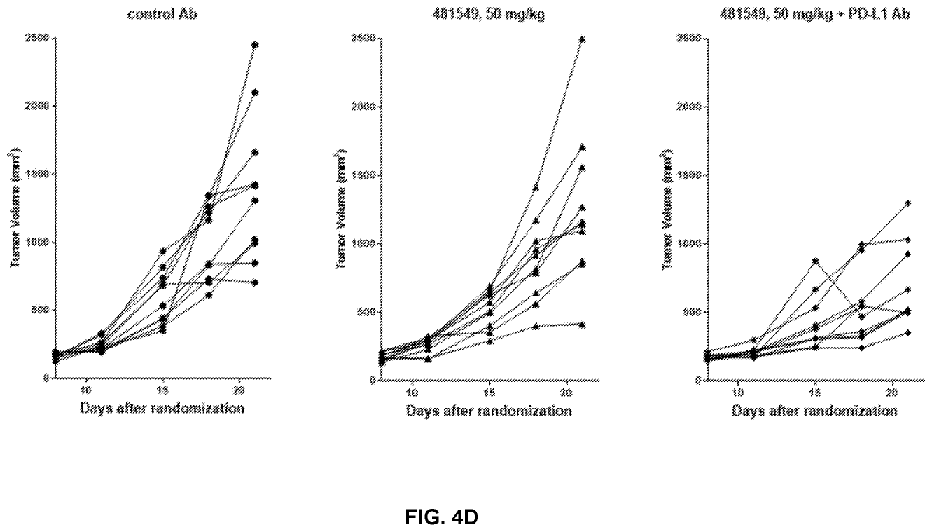

Additional data from experiments testing the anti-PD-L1 antibody and STAT3 ASO, either alone or in combination are shown in FIGS. 4A-4D. FIG. 4B shows tumor growth after treatment of MC38 mice with vehicle, STAT3 ASO, anti-PD-L1 antibody, and STAT3 ASO and anti-PD-L1 antibody. FIG. 4A shows a combination of the data in FIG. 4B. FIG. 4D shows tumor growth after treatment of MC38 mice with control antibody, anti-PD-L1 antibody, STAT3 ASO alone, or a combination of anti-PD-L1 antibody and STAT3 ASO. FIG. 4C shows a combination of the data in FIG. 4D.

Triple-combination-treated mice (i.e., combination of cisplatin, anti-PD-L1 antibody, and STAT3 ASO) showed 20% response rate, while all other treatment groups had 0 complete responses. Flow cytometry studies with the triple-combination-treated mice showed enhanced CD4 T cell functionality (1.6× increase IFNγ, p<0.001, and 1.2× increase IL-2, p=0.001) and enhanced NK functionality (1.3× increase Granzyme B+, p<0.01; 4.3× increase TNFα, p<0.001).

### Sequences

SEQ ID NO: 1 corresponds to a nucleotide sequence of a nucleic acid encoding STAT3 as described in embodiments herein.

SEQ ID NO: 2 corresponds to a nucleotide sequence of AZD9150, which is an antisense compound targeted to STAT3 as described in embodiments herein.

SEQ ID NOs: 3-10 correspond to amino acid sequences of MEDI4736, which is an anti-PD-L1 antibody as described in embodiments herein. SEQ ID NO: 3 corresponds to an amino acid sequence of the light chain variable region of MEDI4736. SEQ ID NO: 4 corresponds to an amino acid sequence of the heavy chain variable region of MEDI4736. SEQ ID NOs: 5-10 correspond to CDRs of MEDI4736.

SEQ ID NO: 11 corresponds to a nucleotide sequence of a mouse STAT3 antisense oligonucleotide as described in embodiments herein.

SEQ ID NO: 12 corresponds to a nucleotide sequence of a control antisense oligonucleotide as described in embodiments herein.

SEQ ID NOs: 13-20 correspond to an amino acid sequence of tremelimumab, which is an anti-CTLA-4 antibody as described in embodiments herein.

SEQ ID NO: 21 corresponds to an amino acid sequence of a CTLA-4 protein as described in embodiments herein.

SEQ ID NO: 22 corresponds to an amino acid sequence of an OX40 protein as described in embodiments herein.

SEQ ID NOs: 23-38 correspond to amino acid or nucleotide sequences of OX40 agonists as described in embodiments herein.

All references cited herein, including patents, patent applications, papers, textbooks and the like, and the references cited therein, to the extent that they are not already, are hereby incorporated herein by reference in their entirety.

What is claimed is:

1. A method of treating cancer in a patient comprising administering to the patient:
a) about 50 mg/m² to about 70 mg/m² chemotherapeutic agent;
b) an immunomodulatory agent; and
c) an antisense compound targeted to STAT3.

2. The method of claim 1, wherein the immunomodulatory agent is an immune checkpoint inhibitor.

3. The method of claim 1 or 2, wherein the immunomodulatory agent is selected from an anti-PD-L1 antibody or antigen-binding fragment thereof; an anti-PD1 antibody or antigen-binding fragment thereof; an anti-CTLA-4 antibody or antigen-binding fragment thereof; and an OX-40 agonist.

4. The method of any of claims 1 to 3, wherein the immunomodulatory agent is selected from MEDI4736, MPDL3280A, 2.7A4, AMP-714, MDX-1105, nivolumab, pembrolizumab, pidilizumab, BMS936559, MPDL3280A, tremelimumab, ipilimumab and OX40L FP.

5. The method of any of claims 1 to 3, wherein the immunomodulatory agent is an anti-PD-L1 antibody.

6. The method of claim 5, wherein the anti-PD-L1 antibody is MEDI4736.

7. The method of any of claims 1 to 6, wherein the antisense compound targeted to STAT3 does not inhibit STAT1, STAT4, or STAT6.

8. The method of any of claims 1 to 7, wherein the antisense compound targeted to STAT3 is an antisense oligonucleotide.

9. The method of any of claims 1 to 8, wherein the antisense compound targeted to STAT3 is AZD9150.

10. The method of any of claims 1 to 9, wherein the immunomodulatory agent is MEDI4736 or an antigen-binding fragment thereof, and the antisense compound targeted to STAT3 is AZD9150.

11. The method of claim 10, comprising administering about 1 mg/kg to about 20 mg/kg MEDI4736 or an antigen-binding fragment thereof.

12. The method of claim 10, comprising administering about 200 mg to about 400 mg AZD9150.

13. The method of any of claims 1 to 12, wherein the chemotherapeutic agent administered to the patient is cisplatin.

14. The method of claim 13, comprising administering about 55 mg/m² to about 65 mg/m² cisplatin.

15. The method of claim 14, comprising administering 60 mg/m² cisplatin.

16. The method of any of claims 1 to 15, wherein the cancer is selected from breast cancer, renal carcinoma, lung cancer, pancreatic cancer, colorectal cancer, hepatocellular carcinoma (HCC), head and neck cancer, and lymphoma.

17. The method of claim 16, wherein the lung cancer is non small cell lung cancer (NSCLC).

18. The method of claim 17, wherein the head and neck cancer is head and neck squamous cell carcinoma (HNSCC).

19. The method of claim 18, wherein the lymphoma is diffuse large B-cell carcinoma (DLBCL).

20. The method of any of claims 1 to 19, wherein the patient has a PD-L1 positive cancer.

21. The method of claim 20, wherein the patient comprises cancer cells expressing PD-L1.

22. The method of any of claims 1 to 21, wherein, in a treatment cycle, the chemotherapeutic agent, the immunomodulatory agent, and the antisense compound targeted to STAT3 are administered to the patient concurrently.

23. The method of any of claims 1 to 21, wherein, in a treatment cycle, the chemotherapeutic agent is administered to the patient before the immunomodulatory agent and the antisense compound targeted to STAT3.

24. The method of any of claims 1 to 21, wherein, in a treatment cycle, the chemotherapeutic agent and the immunomodulatory agent are administered to the patient before the antisense compound targeted to STAT3.

25. The method of any of claims 1 to 21, wherein, in a treatment cycle, fewer doses of the chemotherapeutic agent are administered to the patient than the immunomodulatory agent and the antisense compound targeted to STAT3.

26. The method of claim 25, wherein, in a treatment cycle, about 1 dose of the chemotherapeutic agent, about 2 to about 5 doses of the immunomodulatory agent, and about 5 to about 20 doses of the antisense compound targeted to STAT3 are administered to the patient.

27. The method of any of claims 22 to 26, wherein the treatment cycle is one week, two weeks, three weeks, or four weeks.

28. The method of any of claims 22 to 27, wherein the method comprises two to eight treatment cycles.

29. The method of any of claims 1 to 28, wherein the method results in an increase in CD11b+/Ly6C+ dendritic cells compared to administration of the immunomodulatory agent alone, administration of the antisense compound targeted to STAT3 alone, or administration of the chemotherapeutic agent alone.

30. The method of any of claims 1 to 28, wherein the method results in an increase in progression-free survival and/or overall survival as compared to administration of the immunomodulatory agent alone, administration of the antisense compound targeted to STAT3 alone, or administration of the chemotherapeutic agent alone.

31. A method of treating cancer in a patient comprising administering to the patient:
a) about 50 mg/m² to about 60 mg/m² cisplatin;
b) about 1 mg/kg to about 20 mg/kg MEDI4736; and
c) about 200 mg to about 400 mg AZD9150.

32. The method of claim 31, comprising administering about 60 mg/m² cisplatin, about 10 mg/kg MEDI4736, and about 300 mg AZD9150.

33. A pharmaceutical composition comprising:
a) a chemotherapeutic agent; and
b) an immunomodulatory agent,
wherein the chemotherapeutic agent and the immunomodulatory agent are in the pharmaceutical composition at a weight ratio of about 1:1 to about 1:4.

34. The pharmaceutical composition of claim 33, wherein the chemotherapeutic agent is cisplatin.

35. The pharmaceutical composition of claim 33 or 34, wherein the immunomodulatory agent is an immune checkpoint inhibitor.

36. The pharmaceutical composition of any of claims 33 to 35, wherein the immunomodulatory agent is selected from an anti-PD-L1 antibody or antigen-binding fragment thereof; an anti-PD1 antibody or antigen-binding fragment thereof; an anti-CTLA-4 antibody or antigen-binding fragment thereof; and an OX-40 agonist.

37. The pharmaceutical composition of any of claims 33 to 36, wherein the immunomodulatory agent is selected from MEDI4736, MPDL3280A, 2.7A4, AMP-714, MDX-1105, nivolumab, pembrolizumab, pidilizumab, BMS936559, MPDL3280A, tremelimumab, ipilimumab, and OX40L FP.

38. The pharmaceutical composition of any of claims 33 to 36, wherein the immunomodulatory agent is an anti-PD-L1 antibody.

39. The pharmaceutical composition of claim 38, wherein the anti-PD-L1 antibody is MEDI4736.

40. The pharmaceutical composition of any of claims 33 to 39, wherein the chemotherapeutic agent and the immunomodulatory agent are in the pharmaceutical composition at a weight ratio of about 1:2.

41. A kit for treating cancer, comprising:
a) an chemotherapeutic agent;
b) an immunomodulatory agent; and
c) an antisense compound targeted to STAT3.

42. The kit of claim 41, wherein the chemotherapeutic agent is cisplatin.

43. The kit of claim 41 or 42, wherein the immunomodulatory agent is an immune checkpoint inhibitor.

44. The kit of any of claims 41 to 43, wherein the immunomodulatory agent is selected from an anti-PD-L1 antibody or antigen-binding fragment thereof; an anti-PD1 antibody or antigen-binding fragment thereof; an anti-CTLA-4 antibody or antigen-binding fragment thereof; and an OX-40 agonist.

45. The kit of any of claims 41 to 44, wherein the immunomodulatory agent is selected from MEDI4736, MPDL3280A, 2.7A4, AMP-714, MDX-1105, nivolumab, pembrolizumab, pidilizumab, BMS936559, MPDL3280A, tremelimumab, ipilimumab, and OX40L FP.

46. The kit of any of claims 41 to 44, wherein the immunomodulatory agent is an anti-PD-L1 antibody.

47. The kit of claim 46, wherein the anti-PD-L1 antibody is MEDI4736.

48. The kit of any of claims 41 to 47, wherein the antisense compound targeted to STAT3 does not inhibit STAT1, STAT4, or STAT6.

49. The kit of any of claims 41 to 48, wherein the antisense compound targeted to STAT3 is an antisense oligonucleotide.

50. The kit of any of claims 41 to 49, wherein the antisense compound targeted to STAT3 is AZD9150.

51. The kit of any of claims 41 to 50, wherein the chemotherapeutic agent is cisplatin, the immunomodulatory agent is MEDI4736, and the antisense compound targeted to STAT3 is AZD9150.


SEQUENCE LISTING (38 sequences)

SEQ ID NO: 1 - DNA, 4978 bp, Homo sapiens
ggtttccggagctgcggcggcgcagactgggagggggagccgggggttccgacgtcgcagccgagggaacaagccccaaccggatcctggacaggcaccccggcttggcgctgtctctccccctcggctcggagaggcccttcggcctgagggagcctcgccgcccgtccccggcacacgcgcagccccggcctctcggcctctgccggagaaacagttgggacccctgattttagcaggatggcccaatggaatcagctacagcagcttgacacacggtacctggagcagctccatcagctctacagtgacagcttcccaatggagctgcggcagtttctggccccttggattgagagtcaagattgggcatatgcggccagcaaagaatcacatgccactttggtgtttcataatctcctgggagagattgaccagcagtatagccgcttcctgcaagagtcgaatgttctctatcagcacaatctacgaagaatcaagcagtttcttcagagcaggtatcttgagaagccaatggagattgcccggattgtggcccggtgcctgtgggaagaatcacgccttctacagactgcagccactgcggcccagcaagggggccaggccaaccaccccacagcagccgtggtgacggagaagcagcagatgctggagcagcaccttcaggatgtccggaagagagtgcaggatctagaacagaaaatgaaagtggtagagaatctccaggatgactttgatttcaactataaaaccctcaagagtcaaggagacatgcaagatctgaatggaaacaaccagtcagtgaccaggcagaagatgcagcagctggaacagatgctcactgcgctggaccagatgcggagaagcatcgtgagtgagctggcggggcttttgtcagcgatggagtacgtgcagaaaactctcacggacgaggagctggctgactggaagaggcggcaacagattgcctgcattggaggcccgcccaacatctgcctagatcggctagaaaactggataacgtcattagcagaatctcaacttcagacccgtcaacaaattaagaaactggaggagttgcagcaaaaagtttcctacaaaggggaccccattgtacagcaccggccgatgctggaggagagaatcgtggagctgtttagaaacttaatgaaaagtgcctttgtggtggagcggcagccctgcatgcccatgcatcctgaccggcccctcgtcatcaagaccggcgtccagttcactactaaagtcaggttgctggtcaaattccctgagttgaattatcagcttaaaattaaagtgtgcattgacaaagactctggggacgttgcagctctcagaggatcccggaaatttaacattctgggcacaaacacaaaagtgatgaacatggaagaatccaacaacggcagcctctctgcagaattcaaacacttgaccctgagggagcagagatgtgggaatgggggccgagccaattgtgatgcttccctgattgtgactgaggagctgcacctgatcacctttgagaccgaggtgtatcaccaaggcctcaagattgacctagagacccactccttgccagttgtggtgatctccaacatctgtcagatgccaaatgcctgggcgtccatcctgtggtacaacatgctgaccaacaatcccaagaatgtaaacttttttaccaagcccccaattggaacctgggatcaagtggccgaggtcctgagctggcagttctcctccaccaccaagcgaggactgagcatcgagcagctgactacactggcagagaaactcttgggacctggtgtgaattattcagggtgtcagatcacatgggctaaattttgcaaagaaaacatggctggcaagggcttctccttctgggtctggctggacaatatcattgaccttgtgaaaaagtacatcctggccctttggaacgaagggtacatcatgggctttatcagtaaggagcgggagcgggccatcttgagcactaagcctccaggcaccttcctgctaagattcagtgaaagcagcaaagaaggaggcgtcactttcacttgggtggagaaggacatcagcggtaagacccagatccagtccgtggaaccatacacaaagcagcagctgaacaacatgtcatttgctgaaatcatcatgggctataagatcatggatgctaccaatatcctggtgtctccactggtctatctctatcctgacattcccaaggaggaggcattcggaaagtattgtcggccagagagccaggagcatcctgaagctgacccaggtagcgctgccccatacctgaagaccaagtttatctgtgtgacaccaacgacctgcagcaataccattgacctgccgatgtccccccgcactttagattcattgatgcagtttggaaataatggtgaaggtgctgaaccctcagcaggagggcagtttgagtccctcacctttgacatggagttgacctcggagtgcgctacctcccccatgtgaggagctgagaacggaagctgcagaaagatacgactgaggcgcctacctgcattctgccacccctcacacagccaaaccccagatcatctgaaactactaactttgtggttccagattttttttaatctcctacttctgctatctttgagcaatctgggcacttttaaaaatagagaaatgagtgaatgtgggtgatctgcttttatctaaatgcaaataaggatgtgttctctgagacccatgatcaggggatgtggcggggggtggctagagggagaaaaaggaaatgtcttgtgttgttttgttcccctgccctcctttctcagcagctttttgttattgttgttgttgttcttagacaagtgcctcctggtgcctgcggcatccttctgcctgtttctgtaagcaaatgccacaggccacctatagctacatactcctggcattgcactttttaaccttgctgacatccaaatagaagataggactatctaagccctaggtttctttttaaattaagaaataataacaattaaagggcaaaaaacactgtatcagcatagcctttctgtatttaagaaacttaagcagccgggcatggtggctcacgcctgtaatcccagcactttgggaggccgaggcggatcataaggtcaggagatcaagaccatcctggctaacacggtgaaaccccgtctctactaaaagtacaaaaaattagctgggtgtggtggtgggcgcctgtagtcccagctactcgggaggctgaggcaggagaatcgcttgaacctgagaggcggaggttgcagtgagccaaaattgcaccactgcacactgcactccatcctgggcgacagtctgagactctgtctcaaaaaaaaaaaaaaaaaaaagaaacttcagttaacagcctccttggtgctttaagcattcagcttccttcaggctggtaatttatataatccctgaaacgggcttcaggtcaaacccttaagacatctgaagctgcaacctggcctttggtgttgaaataggaaggtttaaggagaatctaagcattttagacttttttttataaatagacttattttcctttgtaatgtattggccttttagtgagtaaggctgggcagagggtgcttacaaccttgactccctttctccctggacttgatctgctgtttcagaggctaggttgtttctgtgggtgccttatcagggctgggatacttctgattctggcttccttcctgccccaccctcccgaccccagtccccctgatcctgctagaggcatgtctccttgcgtgtctaaaggtccctcatcctgtttgttttaggaatcctggtctcaggacctcatggaagaagagggggagagagttacaggttggacatgatgcacactatggggccccagcgacgtgtctggttgagctcagggaatatggttcttagccagtttcttggtgatatccagtggcacttgtaatggcgtcttcattcagttcatgcagggcaaaggcttactgataaacttgagtctgccctcgtatgagggtgtatacctggcctccctctgaggctggtgactcctccctgctggggccccacaggtgaggcagaacagctagagggcctccccgcctgcccgccttggctggctagctcgcctctcctgtgcgtatgggaacacctagcacgtgctggatgggctgcctctgactcagaggcatggccggatttggcaactcaaaaccaccttgcctcagctgatcagagtttctgtggaattctgtttgttaaatcaaattagctggtctctgaattaagggggagacgaccttctctaagatgaacagggttcgccccagtcctcctgcctggagacagttgatgtgtcatgcagagctcttacttctccagcaacactcttcagtacataataagcttaactgataaacagaatatttagaaaggtgagacttgggcttaccattgggtttaaatcatagggacctagggcgagggttcagggcttctctggagcagatattgtcaagttcatggccttaggtagcatgtatctggtcttaactctgattgtagcaaaagttctgagaggagctgagccctgttgtggcccattaaagaacagggtcctcaggccctgcccgcttcctgtccactgccccctccccatccccagcccagccgagggaatcccgtgggttgcttacctacctataaggtggtttataagctgctgtcctggccactgcattcaaattccaatgtgtacttcatagtgtaaaaatttatattattgtgaggttttttgtcttttttttttttttttttttttggtatattgctgtatctactttaacttccagaaataaacgttatataggaaccgtaaaaa

SEQ ID NO: 2 - DNA, 16 bp, Artificial Sequence
  Synthetic oligonucleotide (AZD9150)
ctatttggatgtcagc

SEQ ID NO: 3 - protein, 108 aa, Artificial Sequence
  MEDI4736 VL; Sequence 77 from PCT/US2010/058007
EIVLTQSPGTLSLSPGERATLSCRASQRVSSSYLAWYQQKPGQAPRLLIYDASSRATGIPDRFSGSGSGTDFTLTISRLEPEDFAVYYCQQYGSLPWTFGQGTKVEIK

SEQ ID NO: 4 - protein, 121 aa, Artificial Sequence
  MEDI4736 VH; Sequence 72 from PCT/US2010/058007
EVQLVESGGGLVQPGGSLRLSCAASGFTFSRYWMSWVRQAPGKGLEWVANIKQDGSEKYYVDSVKGRFTISRDNAKNSLYLQMNSLRAEDTAVYYCAREGGWFGELAFDYWGQGTLVTVSS

SEQ ID NO: 5 - protein, 5 aa, Artificial Sequence
  MEDI4736 VH CDR1; Sequence 73 from PCT/US2010/058007
RYWMS

SEQ ID NO: 6 - protein, 17 aa, Artificial Sequence
  MEDI4736 VH CDR2;Sequence 74 from PCT/US2010/058007
NIKQDGSEKYYVDSVKG

SEQ ID NO: 7 - protein, 12 aa, Artificial Sequence
  MEDI4736 VH CDR3;Sequence 75 from PCT/US2010/058007
EGGWFGELAFDY

SEQ ID NO: 8 - protein, 12 aa, Artificial Sequence
  MEDI4736 VL CDR1;Sequence 78 from PCT/US2010/058007
RASQRVSSSYLA

SEQ ID NO: 9 - protein, 7 aa, Artificial Sequence
  MEDI4736 VL CDR2;Sequence 79 from PCT/US2010/058007
DASSRAT

SEQ ID NO: 10 - protein, 9 aa, Artificial Sequence
  MEDI4736 VL CDR3;Sequence 80 from PCT/US2010/058007
QQYGSLPWT

SEQ ID NO: 11 - DNA, 16 bp, Artificial Sequence
  Synthetic oligonucleotide (murine STAT3 ASO)
gaaattcattcttcca

SEQ ID NO: 12 - DNA, 16 bp, Artificial Sequence
  Synthetic oligonucleotide (murine control ASO)
ggctactacgccgtca

SEQ ID NO: 13 - protein, 139 aa, Artificial Sequence
  Tremelimumab VL
PSSLSASVGDRVTITCRASQSINSYLDWYQQKPGKAPKLLIYAASSLQSGVPSRFSGSGSGTDFTLTISSLQPEDFATYYCQQYYSTPFTFGPGTKVEIKRTVAAPSVFIFPPSDEQLKSGTASVVCLLNNFYPREAKV

SEQ ID NO: 14 - protein, 167 aa, Artificial Sequence
  Tremelimumab VH
GVVQPGRSLRLSCAASGFTFSSYGMHWVRQAPGKGLEWVAVIWYDGSNKYYADSVKGRFTISRDNSKNTLYLQMNSLRAEDTAVYYCARDPRGATLYYYYYGMDVWGQGTTVTVSSASTKGPSVFPLAPCSRSTSESTAALGCLVKDYFPEPVTVSWNSGALTSGVH

SEQ ID NO: 15 - protein, 10 aa, Artificial Sequence
  Tremelimumab VH CDR1
GFTFSSYGMH

SEQ ID NO: 16 - protein, 15 aa, Artificial Sequence
  Tremelimumab VH CDR2
VIWYDGSNKYYADSV

SEQ ID NO: 17 - protein, 16 aa, Artificial Sequence
  Tremelimumab VH CDR3
DPRGATLYYYYYGMDV

SEQ ID NO: 18 - protein, 11 aa, Artificial Sequence
  Tremelimumab VL CDR1
RASQSINSYLD

SEQ ID NO: 19 - protein, 7 aa, Artificial Sequence
  Tremelimumab VL CDR2
AASSLQS

SEQ ID NO: 20 - protein, 9 aa, Artificial Sequence
  Tremelimumab VL CDR3
QQYYSTPFT

SEQ ID NO: 21 - protein, 223 aa, Homo sapiens
MACLGFQRHKAQLNLATRTWPCTLLFFLLFIPVFCKAMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSDFLLWILAAVSSGLFFYSFLLTAVSLSKMLKKRSPLTTGVYVKMPPTEPECEKQFQPYFIPIN

SEQ ID NO: 22 - protein, 277 aa, Artificial Sequence
  OX40 polypeptide
MCVGARRLGRGPCAALLLLGLGLSTVTGLHCVGDTYPSNDRCCHECRPGNGMVSRCSRSQNTVCRPCGPGFYNDVVSSKPCKPCTWCNLRSGSERKQLCTATQDTVCRCRAGTQPLDSYKPGVDCAPCPPGHFSPGDNQACKPWTNCTLAGKHTLQPASNSSDAICEDRDPPATQPQETQGPPARPITVQPTEAWPRTSQGPSTRPVEVPGGRAVAAILGLGLVLGLLGPLAILLALYLLRRDQRLPPDAHKPPGGGSFRTPIQEEQADAHSTLAKI

SEQ ID NO: 23 - protein, 183 aa, Artificial Sequence
  OX40 ligand
MERVQPLEENVGNAARPRFERNKLLLVASVIQGLGLLLCFTYICLHFSALQVSHRYPRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVL

SEQ ID NO: 24 - protein, 410 aa, Artificial Sequence [sequence omitted]

SEQ ID NO: 25 - protein, 107 aa, Artificial Sequence
  humanised anti-OX40 antibody VL
DIQMTQSPSSLSASVGDRVTITCRASQDISNYLNWYQQKPGKAPKLLIYYTSKLHSGVPSRFSGSGSGTDYTLTISSLQPEDFATYYCQQGSALPWTFGQGTKVEIK

SEQ ID NO: 26 - protein, 107 aa, Artificial Sequence
  humanised anti-OX40 antibody VL
DIQMTQSPSSLSASVGDRVTITCRASQDISNYLNWYQQKPGKAVKLLIYYTSKLHSGVPSRFSGSGSRTDYTLTISSLQPEDFATYYCQQGSALPWTFGQGTKVEIK

SEQ ID NO: 27 - protein, 121 aa, Artificial Sequence
  humanised anti-OX40 antibody VH
QVQLQESGPGLVKPSQTLSLTCAVYGGSFSSGYWNWIRKHPGKGLEYIGYISYNGITYHNPSLKSRITINRDTSKNQYSLQLNSVTPEDTAVYYCARYKYDYDGGHAMDYWGQGTLVTVSS

SEQ ID NO: 28 - protein, 451 aa, Artificial Sequence
  humanised anti-Ox40 antibody heavy chain
QVQLQESGPGLVKPSQTLSLTCAVYGGSFSSGYWNWIRKHPGKGLEYIGYISYNGITYHNPSLKSRITINRDTSKNQYSLQLNSVTPEDTAVYYCARYKYDYDGGHAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 29 - protein, 214 aa, Artificial Sequence
  humanised anti-OX40 antibody Light chain
DIQMTQSPSSLSASVGDRVTITCRASQDISNYLNWYQQKPGKAPKLLIYYTSKLHSGVPSRFSGSGSGTDYTLTISSLQPEDFATYYCQQGSALPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 30 - protein, 412 aa, Artificial Sequence
  humanised anti-OX40 antibody
APLATDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKELLGGGSIKQIEDKIEEILSKIYHIENEIARIKKLIGERGHGGGSNSQVSHRYPRFQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVL

SEQ ID NO: 31 - DNA, 1206 bp, Artificial Sequence
  DNA Sequence of huIgG4FcPTF2OX40L
gagagcaagtacggccctccctgccccccttgccctgcccccgagttcctgggcggacctagcgtgttcctgttcccccccaagcccaaggacaccctgatgatcagcagaacccccgaggtgacctgcgtggtggtggacgtgtcccaggaggaccccgaggtccagtttaattggtacgtggacggcgtggaagtgcataacgccaagaccaagcccagagaggagcagttcaacagcacctacagagtggtgtccgtgctgaccgtgctgcaccaggactggctgaacggcaaggaatacaagtgcaaggtctccaacaagggcctgcctagcagcatcgagaagaccatcagcaaggccaagggccagccacgggagccccaggtctacaccctgccacctagccaagaggagatgaccaagaaccaggtgtccctgacctgtctggtgaaaggcttctatcccagcgatatcgccgtggagtgggagagcaacggccagcccgagaacaactacaagaccaccccccctgtgctggacagcgacggcagcttcttcctgtactccagactgaccgtggacaagtccagatggcaggagggcaacgtcttcagctgctccgtgatgcacgaggccctgcacaaccactacacccagaagtccctgagcctgagcctgggcaaggaccaggataagatcgaggctctgtcctccaaggtgcagcagctggaacggtccatcggcctgaaggacctggccatggctgacctggaacagaaagtgctggaaatggaagcctccacacaggtgtcacacagatacccccggatccagtccattaaggtgcagttcaccgagtacaagaaagagaagggctttatcctgacctcccagaaagaggacgagatcatgaaggtgcagaacaactccgtgatcatcaactgcgacgggttctacctgatctccctgaagggctacttcagccaggaagtgaacatctccctgcactaccagaaggacgaggaacccctgttccagctgaagaaagtgcggagcgtgaactccctgatggtggcctctctgacctacaaggacaaggtgtacctgaacgtgaccaccgacaacacctccctggacgacttccacgtgaacggcggcgagctgatcctgatccaccagaaccctggcgagttctgcgtgctg

SEQ ID NO: 32 - protein, 402 aa, Artificial Sequence
  Amino Acid Sequence of huIgG4FcPTF2OX40L
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKDQDKIEALSSKVQQLERSIGLKDLAMADLEQKVLEMEASTQVSHRYPRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVL

SEQ ID NO: 33 - DNA, 1206 bp, Artificial Sequence
  DNA Sequence of huIgG4FcPTF2OX40L F180A
gagagcaagtacggccctccctgccccccttgccctgcccccgagttcctgggcggacctagcgtgttcctgttcccccccaagcccaaggacaccctgatgatcagcagaacccccgaggtgacctgcgtggtggtggacgtgtcccaggaggaccccgaggtccagtttaattggtacgtggacggcgtggaagtgcataacgccaagaccaagcccagagaggagcagttcaacagcacctacagagtggtgtccgtgctgaccgtgctgcaccaggactggctgaacggcaaggaatacaagtgcaaggtctccaacaagggcctgcctagcagcatcgagaagaccatcagcaaggccaagggccagccacgggagccccaggtctacaccctgccacctagccaagaggagatgaccaagaaccaggtgtccctgacctgtctggtgaaaggcttctatcccagcgatatcgccgtggagtgggagagcaacggccagcccgagaacaactacaagaccaccccccctgtgctggacagcgacggcagcttcttcctgtactccagactgaccgtggacaagtccagatggcaggagggcaacgtcttcagctgctccgtgatgcacgaggccctgcacaaccactacacccagaagtccctgagcctgagcctgggcaaggaccaggataagatcgaggctctgtcctccaaggtgcagcagctggaacggtccatcggcctgaaggacctggccatggctgacctggaacagaaagtgctggaaatggaagcctccacacaggtgtcacacagatacccccggatccagtccattaaggtgcagttcaccgagtacaagaaagagaagggctttatcctgacctcccagaaagaggacgagatcatgaaggtgcagaacaactccgtgatcatcaactgcgacgggttctacctgatctccctgaagggctacttcagccaggaagtgaacatctccctgcactaccagaaggacgaggaacccctgttccagctgaagaaagtgcggagcgtgaactccctgatggtggcctctctgacctacaaggacaaggtgtacctgaacgtgaccaccgacaacacctccctggacgacttccacgtgaacggcggcgagctgatcctgatccaccagaaccctggcgaggcctgcgtgctg

SEQ ID NO: 34 - protein, 1206 aa, Artificial Sequence
  Amino Acid Sequence of huIgG4PFcTF2OX40L F180A
GAGAGCAAGTACGGCCCTCCCTGCCCCCCTTGCCCTGCCCCCGAGTTCCTGGGCGGACCTAGCGTGTTCCTGTTCCCCCCCAAGCCCAAGGACACCCTGATGATCAGCAGAACCCCCGAGGTGACCTGCGTGGTGGTGGACGTGTCCCAGGAGGACCCCGAGGTCCAGTTTAATTGGTACGTGGACGGCGTGGAAGTGCATAACGCCAAGACCAAGCCCAGAGAGGAGCAGTTCAACAGCACCTACAGAGTGGTGTCCGTGCTGACCGTGCTGCACCAGGACTGGCTGAACGGCAAGGAATACAAGTGCAAGGTCTCCAACAAGGGCCTGCCTAGCAGCATCGAGAAGACCATCAGCAAGGCCAAGGGCCAGCCACGGGAGCCCCAGGTCTACACCCTGCCACCTAGCCAAGAGGAGATGACCAAGAACCAGGTGTCCCTGACCTGTCTGGTGAAAGGCTTCTATCCCAGCGATATCGCCGTGGAGTGGGAGAGCAACGGCCAGCCCGAGAACAACTACAAGACCACCCCCCCTGTGCTGGACAGCGACGGCAGCTTCTTCCTGTACTCCAGACTGACCGTGGACAAGTCCAGATGGCAGGAGGGCAACGTCTTCAGCTGCTCCGTGATGCACGAGGCCCTGCACAACCACTACACCCAGAAGTCCCTGAGCCTGAGCCTGGGCAAGGACCAGGATAAGATCGAGGCTCTGTCCTCCAAGGTGCAGCAGCTGGAACGGTCCATCGGCCTGAAGGACCTGGCCATGGCTGACCTGGAACAGAAAGTGCTGGAAATGGAAGCCTCCACACAGGTGTCACACAGATACCCCCGGATCCAGTCCATTAAGGTGCAGTTCACCGAGTACAAGAAAGAGAAGGGCTTTATCCTGACCTCCCAGAAAGAGGACGAGATCATGAAGGTGCAGAACAACTCCGTGATCATCAACTGCGACGGGTTCTACCTGATCTCCCTGAAGGGCTACTTCAGCCAGGAAGTGAACATCTCCCTGCACTACCAGAAGGACGAGGAACCCCTGTTCCAGCTGAAGAAAGTGCGGAGCGTGAACTCCCTGATGGTGGCCTCTCTGACCTACAAGGACAAGGTGTACCTGAACGTGACCACCGACAACACCTCCCTGGACGACTTCCACGTGAACGGCGGCGAGCTGATCCTGATCCACCAGAACCCTGGCGAGGCCTGCGTGCTG

SEQ ID NO: 35 - DNA, 1200 bp, Artificial Sequence
  DNA Sequence of huIgG1TF2OX40L
gataagacccacacctgtcccccttgtcctgcccctgaactgctgggcggaccttccgtgttcctgttccccccaaagcccaaggacaccctgatgatctcccggacccccgaagtgacctgcgtggtggtggatgtgtcccacgaggaccctgaagtgaagttcaattggtacgtggacggcgtggaagtgcacaacgccaagaccaagcccagagaggaacagtacaactccacctaccgggtggtgtccgtgctgaccgtgctgcaccaggattggctgaacggcaaagagtacaagtgcaaggtgtccaacaaggccctgcctgcccccatcgaaaagaccatctccaaggccaagggccagccccgggaaccccaggtgtacacactgccccctagccgggaagagatgaccaagaaccaggtgtccctgacctgtctcgtgaagggcttctacccctccgatatcgccgtggaatgggagtccaacggccagcctgagaacaactacaagaccaccccccctgtgctggactccgacggctcattcttcctgtactccaagctgacagtggacaagtcccggtggcagcagggcaacgtgttctcctgctccgtgatgcacgaggccctgcacaaccactacacccagaagtccctgtccctgagccccggcaaggaccaggataagatcgaggctctgtcctccaaggtgcagcagctggaacggtccatcggcctgaaggacctggccatggctgacctggaacagaaagtgctggaaatggaagcctccacacaggtgtcacacagatacccccggatccagtccattaaggtgcagttcaccgagtacaagaaagagaagggctttatcctgacctcccagaaagaggacgagatcatgaaggtgcagaacaactccgtgatcatcaactgcgacgggttctacctgatctccctgaagggctacttcagccaggaagtgaacatctccctgcactaccagaaggacgaggaacccctgttccagctgaagaaagtgcggagcgtgaactccctgatggtggcctctctgacctacaaggacaaggtgtacctgaacgtgaccaccgacaacacctccctggacgacttccacgtgaacggcggcgagctgatcctgatccaccagaaccctggcgagttctgcgtgctg

SEQ ID NO: 36 - protein, 400 aa, Artificial Sequence
  Amino Acid Sequence of huIgG1FcTF2OX40L
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKDQDKIEALSSKVQQLERSIGLKDLAMADLEQKVLEMEASTQVSHRYPRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVL

SEQ ID NO: 37 - DNA, 1248 bp, Artificial Sequence
  DNA Sequence of mouse construct mIgG1mTF2mOX40L
gtgcctagagattgcggctgcaagccctgcatctgcaccgtgcccgaggtgtccagcgtgttcatcttcccacccaagcccaaggacgtgctgaccatcaccctgacccccaaagtgacctgcgtggtggtggacatcagcaaggacgaccccgaggtgcagttcagttggttcgtggacgacgtggaagtgcacaccgcccagacccagcccagagaggaacagttcaacagcaccttcagaagcgtgtccgagctgcccatcatgcaccaggactggctgaacggcaaagaattcaagtgcagagtgaacagcgccgccttccctgcccccatcgagaaaaccatcagcaagaccaagggcagacccaaggccccccaggtgtacaccatccccccacccaaagaacagatggccaaggacaaggtgtccctgacctgcatgatcaccgatttcttcccagaggacatcaccgtggaatggcagtggaacggccagcccgccgagaactacaagaacacccagcccatcatggacaccgacggcagctacttcgtgtacagcaagctgaacgtgcagaagtccaactgggaggccggcaacaccttcacctgtagcgtgctgcacgagggcctgcacaaccaccacaccgagaagtccctgagccacagccccggcaagcggctggaccaggacaagatcgaggccctgagcaacaaggtgcagcagctggaacggtctatcggcctgaaggacctggctatggccgacctggaacagaaagtgtctgagctggaagtgtccaccagcagccccgccaaggaccctcccatccagagactgagaggcgccgtgaccagatgcgaggacggccagctgttcatcagcagctacaagaacgagtaccagaccatggaagtgcagaacaacagcgtggtcatcaagtgcgacggcctgtacatcatctacctcaagggcagcttcttccaggaagtgaagatcgacctgcacttcagagaggaccacaaccccatcagcatccccatgctgaacgacggcagacggatcgtgttcaccgtggtggctagcctggccttcaaggacaaagtgtatctgaccgtgaacgcccccgacaccctgtgcgagcatctgcagatcaacgacggcgagctgatcgtggtgcagctgacccccggctactgtgcccctgagggcagctaccacagcaccgtgaaccaggtgcccctg

SEQ ID NO: 38 - protein, 416 aa, Artificial Sequence
  Amino Acid Sequence of mouse construct mIgG1FcmTF2mOX40L
VPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGKRLDQDKIEALSNKVQQLERSIGLKDLAMADLEQKVSELEVSTSSPAKDPPIQRLRGAVTRCEDGQLFISSYKNEYQTMEVQNNSVVIKCDGLYIIYLKGSFFQEVKIDLHFREDHNPISIPMLNDGRRIVFTVVASLAFKDKVYLTVNAPDTLCEHLQINDGELIVVQLTPGYCAPEGSYHSTVNQVPL